(19) United States
(12) Patent Application Publication
(10) Pub. No.: US 2015/0237857 A1
(43) Pub. Date: Aug. 27, 2015

(54) ANTIBACTERIAL INHIBITORS

(71) Applicant: MCMASTER UNIVERSITY, Hamilton (CA)

(72) Inventors: Eric Brown, Oakville (CA); Soumaya Alitni, Hamilton (CA)

(21) Appl. No.: 14/420,846

(22) PCT Filed: Aug. 9, 2013
(86) PCT No.: PCT/CA2013/000709

§ 371 Date: Feb. 10, 2015

Related U.S. Application Data
(60) Provisional application No. 61/681,674, filed on Aug. 10, 2012.

(51) Int. Cl. A01N 43/40; A01N 37/38; C07C 323/22; C07D 213/04; C07C 323/62; C07D 333/70; A01N 43/12; C07C 323/48; A01N 35/02

(52) U.S. Cl. CPC A01N 43/40; C07C 323/48; A01N 37/38; C07C 323/22; A01N 35/02; C07C 323/62; C07D 333/70; A01N 43/12; C07D 213/04

(57) ABSTRACT

Anti-bacterial compounds have been identified having the general structures defined by Formula (I), Formula (II), and Formula (III).

### FIELD OF INVENTION

The present invention relates to anti-bacterial compounds.

### BACKGROUND OF THE INVENTION

The alarming spread of multidrug resistance is due in part to the fact that existing antibiotics target a very limited number of pathways, namely pathways involved in cell wall, DNA and protein biosynthesis. In fact, in the past forty years only two new chemical classes of antibiotics, the oxazolidinone, linezolid, and the lipopeptide, daptomycin, have been introduced for clinical use, while considerable efforts in antibacterial drug discovery have focused on chemically modifying existing antibiotic scaffolds.

The vast developments in microbial genomics, target validation and screening technology have provided drug discoverers with many avenues to identify novel antibacterial leads. Moreover, given the significant challenges faced when attempting to convert target-specific leads into cell-active compounds, recent antibacterial drug discovery campaigns have shifted towards phenotype-based screening to identify the next generation of antibiotics. However, many of these efforts have been halted by the difficulty in identifying the mode of inhibition of newly discovered leads. Indeed, linking the phenotype(s) caused by biologically active small molecules to specific mechanisms remains one of the biggest roadblocks in cell-based screening. What further plagues the process is the general lack of systematic approaches to address this central question.

In this respect, chemical genomic strategies have had considerable success in shedding light on the mechanism of action of biologically active molecules. Most significant are the efforts in the characterization of the mode of action of small molecules by exploring their effects on genome-scale overexpression and deletion clone sets.

However, there remains a need to develop novel antibacterial treatments.

### SUMMARY OF THE INVENTION

Compounds having antibacterial activity have been identified using differential media screening and metabolite chemical suppression.

Thus, in one aspect of the invention, the use of a compound of formula (I) as an anti-bacterial agent is provided:

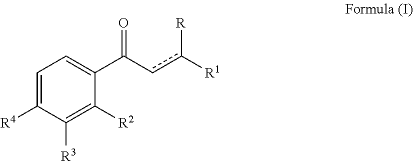

In another aspect of the invention, the use of a compound of formula (II) as an anti-bacterial agent is provided:

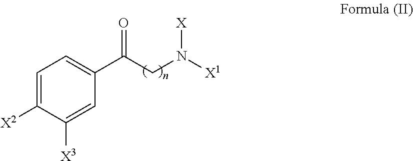

In a further aspect of the invention, the use of a compound of formula (III) as an anti-bacterial agent is provided:

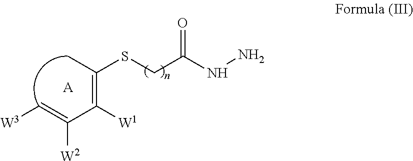

These and other aspects of the invention are described by reference to the detailed description and figures which follow.

### BRIEF DESCRIPTION OF THE FIGURES

FIG. 1 illustrates a flow chart (a) of the methods used to characterize inhibitors of bacterial physiology under nutrient-limited growth conditions to identify priority actives, and the chemical structures of priority actives (b);

FIG. 2 graphically illustrates the effect of media composition on the EC50 of MAC-0006191 which has an EC50 of 0.5 μM in minimal media (a) but no growth inhibitory activity in supplemented minimal media (b); and on MAC-0043268 which has EC50 values of 0.26 (c) and 6.86 μM (d) when tested in minimal and supplemented minimal media, respectively.

FIG. 3 is a histogram of the average EC50 values obtained from the dose-response analysis of 340 novel bioactives conducted in minimal (black bars) and supplemented minimal media (grey bars);

FIG. 4 illustrates the chemical structures of a cluster of compounds that are suppressed by p-aminobenzoic acid;

FIG. 5 illustrates the chemical structure of MAC-0173979, an inhibitor of p-aminobenzoic acid biosynthesis in E. Coli (a); progress curves of the production of PABA in the presence of various concentrations of MAC-0173979 (b); a plot of k_{obs} as a function of [MAC-0173979] indicating an irreversible time-dependent mechanism of inhibition (c); a metabolic suppression profile of MAC-0173979 (M9: minimal media, no supplements; ALL: minimal media with all supplements; AA: minimal media with all amino acids; VIT: minimal media with all vitamins; NUC: minimal media with all nucleobases) (d); analogues of MAC-0173979 (e); and dose-response curves for MAC-0173979 and an analogue lacking the Michael acceptor (f);

FIG. 6 illustrates the chemical structure of MAC-0168425 (a); a graph illustrating that the minimum inhibitory concentration (MIC) of MAC-0168425 increases in the presence of increasing concentrations of L-threonine (b) and analogues of MAC-0168425 (c);

FIG. 7 is a metabolic suppression profile of MAC-0168425; and

FIG. 8 graphically illustrates the metabolic suppression profile of MAC-0013772 (a); dose-response curve of MAC-0013772 against recombinant BioA (b); spectral analysis of the BioA-MAC-0013772 interaction (c); and a proposed model for BioA-MAC-0013772 interaction.

### DETAILED DESCRIPTION OF THE INVENTION

In a first aspect, the use of a compound of formula (I) as an anti-bacterial agent is provided:

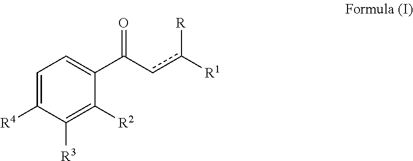

wherein
R and R¹ are independently selected from H, C₁-C₆ alkyl, C₁-C₆ alkyl halide, halogen (e.g. Br, Cl, F and I), hydroxyl, thiol, carboxyl, acyl halide (—CO-halogen), alkanoyl (—COR^{a}), —OR^{a}, —NH₂, —NO₂, —NHR^{a}, —NR^{a}R^{b} or —SR^{a}, wherein R^{a} and R^{b} are independently selected from C₁-C₆ alkyl; and
R², R³ and R⁴ are independently selected from H, C₁-C₆ alkyl, C₁-C₆ alkyl halide, halogen, hydroxyl, thiol, carboxyl, acyl halide, alkanoyl (—COR^{a}), —OR^{a}, —NH₂, —NO₂, —NHR^{a}, —NR^{a}R^{b} or —SR^{a}, wherein R^{a} and R^{b} are independently selected from C₁-C₆ alkyl.

For clarity, C₁-C₆ alkyl includes linear and branched alkyl groups. Examples of suitable alkyl groups include, ut are not limited to, methyl, ethyl, propyl, isopropyl, butyl, isobutyl, sec-butyl, tert-butyl, pentyl, isopentyl, 3-methylpentyl, hexyl and isohexyl.

In one embodiment, R and R¹ in compounds of Formula (I) are the same substituent, eg. both are halogen, hydroxyl, alkyl, or other substituents. In another embodiment, one of R², R³ and R⁴ is C₁-C₆ alkyl, C₁-C₆ alkyl halide, halogen, hydroxyl, thiol, carboxyl, acyl halide, alkanoyl (—COR^{a}), —OR^{a}, —NH₂, —NO₂, —NHR^{a}, —NR^{a}R^{b} or —SR^{a}, while the other two of R², R³ and R⁴ are each H, for example, R³ is —NO₂ and R² and R⁴ are each H.

Examples of compounds within Formula (I) include 3,3-dichloro-1-(3-nitrophenyl)prop-2-en-1-one (referred to herein as MAC173979), 3,3-dichloro-1-(3-nitrophenyl)propan-1-one (analog of MAC173979 analog without the Michael acceptor), 1-(3-nitrophenyl)propan-1-one and 3-methyl-1-(3-nitrophenyl)butan-1-one. Such compounds may be purchased, or chemically synthesized using well-established synthetic techniques.

In another aspect, the use of a compound of formula (II) as an anti-bacterial agent is provided:

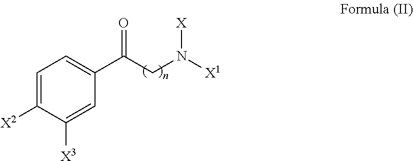

wherein
X and X¹ are independently selected from H, C₁-C₆ alkyl, C₁-C₆ alkyl halide, carboxyl, acyl halide, —COR^{a}, —COOR^{a} and C₁-C₆ alkyl-carboxyl, wherein R^{a} and R^{b} are independently selected from C₁-C₆ alkyl; or

X and X¹ together form a heterocyclic ring with N₁, wherein said ring comprises from 4-6 carbon atoms and may include a second hetero atom selected from N or S, and wherein said ring is optionally substituted with a group selected from C₁-C₆ alkyl, C₁-C₆ alkyl halide, halogen, hydroxyl, carboxyl, acyl halide, —COR^{a}, —COOR^{a} and C₁-C₆ alkyl-carboxyl, wherein R^{a} and R^{b} are independently selected from C₁-C₆ alkyl;

X² and X³ are independently selected from H, C₁-C₆ alkyl, C₁-C₆ alkyl halide, halogen, hydroxyl, thiol, carboxyl, acyl halide, —COR^{a}, —OR^{a}, —NH₂, —NO₂, —NHR^{a}, —NR^{a}R^{b} or —SR^{a}, wherein R^{a} and R^{b} are independently selected from C₁-C₆ alkyl; or

X² and X³ together form a ring, wherein said ring may be a heterocyclic ring comprising 1 or 2 hetero atoms selected from O or N, and said ring structure may be optionally substituted with a group selected from C₁-C₆ alkyl, C₁-C₆ alkyl halide, halogen, hydroxyl, carboxyl, acyl halide, —COR^{a}, —COOR^{a} and C₁-C₆ alkyl-carboxyl, wherein R^{a} and R^{b} are independently selected from C₁-C₆ alkyl; and n is 1-5.

Examples of heterocyclic rings that may be formed by X and X¹ include 5- to 8-membered ring structures such as, but not limited to, pyrrole, pyrrolidine, pyrimidine, piperazine, piperadine, pyridine, diazine, azepane, azepine, azopane, azocane and azocine.

Examples of heterocyclic rings formed by X² and X³ include dioxolane, tetrahydrofuran, furan, oxane, dioxane, oxapane, oxepine, dioxapane, dioxapine, thiane, thiopyran, dithiane, dithiine, thiepane, thiolane and thiophene.

Examples of compounds within Formula (II) include 3-(dimethylamino)-1-(4-methoxyphenyl)propan-1-one ((MAC168425), 1-(2,3-dihydrobenzo[b][1,4]dioxin-6-yl)-3-(dimethylamino)propan-1-one (MAC161738), and 1-(3,4-dihydro-2H-benzo[b][1,4]dioxepin-7-yl)-3-(piperidin-1-yl)propan-1-one) (MAC162065). Such compounds may be purchased, or chemically synthesized using well-established synthetic techniques.

In a further aspect, the use of a compound of formula (III) as an anti-bacterial agent is provided:

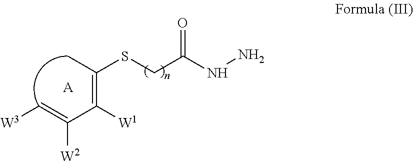

wherein
A is a ring selected from phenyl, pyridinyl, naphthanyl, quinoline and indole;
W¹, W² and W³ are independently selected from is H, OH, NO₂, NH₂, halogen (e.g. F, Cl, Br and I), C₁-C₆ alkyl, C₁-C₆ alkyl halide, carboxyl, acyl halide, COR^{a}, wherein R^{a} and R^{b} are independently selected from C₁-C₆ alkyl; and
n is 0-5.

Examples of compounds within Formula (III) include 2-(2-nitrophenylthio) acetohydrazide, 2-(3-nitrophenylthio)acetohydrazide, 2-(4-nitrophenylthio)acetohydrazide, 2-(phenylthio)acetohydrazide, 2-(2-fluorophenylthio)acetohydrazide, 2-(2-chlorophenylthio) acetohydrazide, 2-(2-hydroxyphenylthio)acetohydrazide, 2-(2-aminophenylthio)acetohydrazide, 2-(o-tolylthio)acetohydrazide, 2-(2-methoxyphenylthio)acetohydrazide, 2,3-dihydrobenzo[b]thiophene-2-carbohydrazide, 2-(benzylthio)acetohydrazide, 2-(pyridin-4-ylthio)acetohydrazide and 2-(naphthalen-2-ylthio)acetohydrazide. Such compounds may be purchased, or chemically synthesized using well-established synthetic techniques.

In an embodiment, the compound of formula (III) may have the following structure:

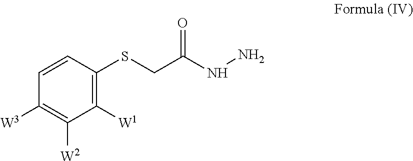

wherein W¹, W² and W³ are as defined above.

In one embodiment, one of W¹, W² and W³ is OH, NO₂, NH₂, halogen, C₁-C₆ alkyl, C₁-C₆ alkyl halide, carboxyl, acyl halide, COR^{a}, wherein R^{a} and R^{b} are independently selected from C₁-C₆ alkyl, while the other two of W¹, W² and W³ are each H.

Compounds of general formula (I), (II) and (III) are useful as antibacterial agents. The term “antibacterial” is used herein to refer to an agent that at least inhibits the growth of or kills one or more bacteria. The compounds are not particularly restricted with respect to the bacteria against which they are active. Examples of target bacteria include pathogenic bacteria such as Escherichia coli, Enterrococci such as Enterococcus faecalis and Enterococcus faecium, Streptococcus such as S. pneumoniae, S. viridans S. pyogenes and S. pharyngitis, Staphylococcus such as S aureus, Pseudomonas such as P. aeruginosa and P. syringae, Salmonella such as S. enterica, S. typhi and S. panama, Mycobacteria such as M. tuberculosis, M. bovis, M. africanum, M. microti and M. leprae, Acinebacter such as Acinetobacter baumannii and Klebsiella such as Klebsiella pneumonia, K. granulomatis and K. planticola.

Compounds within the scope of general formula (I), (II) and (III) and which exhibit anti-bacterial activity may readily be identified using standard assays as described herein to determine their minimal inhibitory concentration (MIC) against bacterial growth. Compounds having an MIC of no more than about 50 μg/ml, for example no more than about 30-40 μg/ml, including MIC of 25 μg/ml or less, e.g. between about 10-20 μg/ml or less, are candidate anti-bacterial compounds in accordance with an aspect of the present invention.

While not wishing to be limited by any particular mode of action, metabolic suppression profiling of the compounds of Formula (I) has revealed that these compounds inhibit bacterial p-aminobenzoic acid biosynthesis. Metabolic suppression profiling of the compounds of Formula (II) has revealed that these compounds inhibit glycine metabolism in bacteria. Metabolic suppression profiling of the compounds of Formula (III) reveal that these compounds inhibit bacterial biotin synthesis. As a result, these compounds each have a broad spectrum of utility as they interfer with pathways common to most bacteria.

As one of skill in the art will appreciate, compounds in accordance with the present invention may be utilized in the form of a salt, hydrate or solvate which is functionally equivalent to the parent compound and which is also pharmaceutically or agriculturally acceptable. The term “functionally equivalent” refers to a salt, hydrate or solvate that retains the desired biological activity of the parent compound, although the activity need not be at the same level of the parent compound. Preferably, the activity of a functionally equivalent salt, hydrate or solvate is at least about 50% of the parent compound, for example, at least 60%, 70%, 80%, 90% or greater. The term “pharmaceutically acceptable” refers to a salt, hydrate or solvate that is acceptable for use in the pharmaceutical arts, i.e. not being unacceptably toxic, or otherwise unsuitable for administration to a mammal, while the term “agriculturally acceptable” indicates acceptability for use in the agricultural arts, i.e. not being unacceptably adverse to an agricultural environment, e.g. unacceptably toxic, or otherwise unsuitable for agricultural use. Examples of suitable salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as those derived from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. A “solvate” is formed by admixture of the compound in a solvent which is preferably pharmaceutically acceptable. A “hydrate” is formed by combination of the compound with water.

The present compounds may be combined with one or more pharmaceutically acceptable adjuvants or carriers for use in the treatment of a mammalian bacterial infection. Examples of pharmaceutically acceptable adjuvants include, but are not limited to, diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule, lozenge, solution or suspension in an aqueous or non-aqueous liquid, an oil-in-water or water-in-oil liquid emulsion, an elixir or syrup are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, disintegrating agents, colouring agents and flavouring agents may also be present. In another embodiment, the composition may be formulated for application topically as a cream, lotion or ointment. For such topical application, the composition may include an appropriate base such as a triglyceride base. Such creams, lotions and ointments may also contain a surface-active agent and other cosmetic additives such as skin softeners and the like as well as fragrance. Aerosol formulations, for example, for nasal delivery, may also be prepared in which suitable propellant adjuvants are used. Compositions of the present invention may also be administered as a bolus, electuary, or paste. Compositions for mucosal administration are also encompassed, including oral, nasal, rectal or vaginal administration for the treatment of infections, which affect these areas. Such compositions generally include one or more suitable non-irritating excipients or carriers comprising, for example, cocoa butter, polyethylene glycol, a suppository wax, a salicylate or other suitable carriers. Other adjuvants may also be added to the composition regardless of how it is to be administered, which, for example, may aid to extend the shelf-life thereof, including anti-oxidants, preservatives, anti-microbial agents and the like.

For use in treating agricultural materials infected with a bacterial pathogen, an anti-bacterial compound may be combined with one or more adjuvants suitable for agricultural use. The term “agricultural materials” is used herein to refer to plants and plant material such as natural or artificial seeds, asexual plant propagation material, such as root, stem or leaf cuttings, tubers, bulbs, corms, rhizomes, and plant micropropagation material such as plant tissue culture and somatic embryonic material, as well as soil, liquids used for irrigation, fertilizers and the like. Adjuvants suitable for agricultural use include, but are not limited to, water, soil, sand, cellulose, peat, plant growth additives, fertilizers, binders such as triglyceride based plant oils, e.g. soya, canola, sunflower, corn or olive oils; thickening agents such as plant isolates, e.g. guar gum, acacia gum, tragacanth, arabic gum, gluten, pectin, starch, carrageenan, agars, cellulose and hemi-cellulose based thickeners, animal isolates such as gelatin and microbial isolates such as xanthan gum, glomalin and glomalin-like proteins.

The present compounds, either alone or in the form of a composition, are useful to treat a bacterial infection, either in a mammal, or an agricultural-based bacterial infection. The terms “treat”, “treating” and “treatment” are used broadly herein to denote methods that at least reduce one or more adverse affects of a bacterial infection, including those that moderate or reverse the progression of, reduce the severity of, prevent, or cure the infection. The term “mammal” as it is used herein is meant to encompass humans as well as non-human mammals such as domestic animals (e.g. dogs, cats and horses), livestock (e.g. cattle, pigs, goats, sheep) and wild animals.

For use to treat a bacterial infection in a mammal, a selected compound having a general formula (I), (II) or (III) is administered to the mammal. The compound may be administered via any suitable route. As will be appreciated by the skilled artisan, the route and/or mode of administration may vary on a number of factors, including for example, the compound to be administered, and the mammal and infection to be treated. Routes of administration include parental, such as intravenous, intramuscular, intradermal, intraperitoneal, subcutaneous, spinal, intraarterial, intrathecal, intracapsular, intraorbital, intracardiac, transtracheal, subcuticular, intraarticular, subcapsular, subarachnoid, intraspinal, epidural and intrasternal injection and infusion. Alternatively, non-parenteral routes may be used, including topical, epidermal or mucosal routes of administration, for example, intranasally, orally, vaginally, rectally, sublingually or topically.

For use to treat a bacterial infection in agricultural material, a selected compound having a general formula (I), (II) or (III) is administered to the agricultural material using well-established techniques for such administration. The compound may be administered directly to the agricultural material in a composition or innoculum as described, or it may be administered to the growth medium of the agricultural material, e.g. soil or other medium in which the material is growing.

The selected compound is administered in the treatment of a bacterial infection using a “therapeutically effective dosage”, i.e. a dosage that is effective to treat a target bacterial infection. As one of skill in the art will appreciate, effective dosage levels will vary with factors such as the pathogenic organism (or type of infection), the compounds selected for use, the mammal or material being treated and mode of administration. Therapeutically effective dosages of the present anti-bacterial compounds is a dosage that would achieve drug concentrations in the range of the MIC of the compound at the site of infection, for example, a dosage of between about 1-300 mg.

An article of manufacture is also provided in another aspect of the invention. The article of manufacture comprises packaging and a composition comprising a compound having the general formula of Formula (I), (II) or (III), wherein the packaging is labelled to indicate that the composition is for use as an anti-bacterial, to inhibit bacterial growth.

Embodiments of the invention are described by reference to the following specific examples which are not to be construed as limiting.

### EXAMPLES

### Example 1

### Bacterial Strains, Reagents and General Methods

E. coli was grown at 37° C. in liquid M9 minimal media with aeration at 250 rpm or on solid M9 agar supplemented with appropriate antibiotics unless otherwise mentioned. The concentration of antibiotics for selection was as follows: 100 μg/ml ampicillin, 30 μg/ml chloramphenicol and 30 μg/ml kanamycin. All of the library compounds were solubilized in DMSO. For the primary screen, the CCC library compounds were prepared to a final concentration of 250 μM in 25% DMSO. When required, arabinose was added at a final concentration of 0.2% (wt/vol) and IPTG was added at a final concentration of 0.1 mM unless otherwise mentioned. All compounds were dissolved in DMSO. All chemicals were purchased from Sigma (Oakville, ON).

### Primary Screen in Minimal Media

A single colony of E. coli MG1655 was grown overnight in M9 minimal media in a 37° C. incubator shaking at 250 rpm. The saturated overnight culture was diluted 1/50 in fresh M9 minimal media and grown in a 37° C. incubator shaking at 250 rpm until it reached an OD600 of ˜0.5. The clear flat bottom 96-well assay plates were set up with the CCC library compounds in triplicate to a final concentration of 10 μM and with high and low controls of 0.2% DMSO and 10 μg/ml of norfloxacin, respectively. Controls constituted 20% of each assay plate. All the liquid handling was carried out using the Biomek FX liquid handler (Beckman/Coulter). The mid-log subculture was then diluted 103-fold into fresh M9 minimal media and set up in the assay plates using the gill Microplate Dispenser (Biotek) to a final volume of 200 μl per well. Upon mixing of the bacterial culture with the screening compounds, the OD600 of the plates was read using the Envision (Perkin Elmer). This background reading is especially useful to account for any interference due to low compound solubility in the growth media or due to colored compounds. The plates were then incubated in a 37° C. stationary incubator for 12 hours before measuring their OD600.

### Dose-Response Determination of Priority Bioactives

The 11-point dose-response determinations were carried out in duplicate in two types of media: M9 minimal media and the same media supplemented with amino acids, vitamins and nucleobases. Briefly, a single colony of E. coli MG1655 was grown overnight in M9 minimal media in a 37° C. incubator shaking at 250 rpm. The saturated overnight culture was diluted 1/50 in fresh M9 minimal media and grown in a 37° C. incubator shaking at 250 rpm until it reached an OD600 of ˜0.5. The subculture was then diluted 103-fold into either fresh M9 minimal media or supplemented M9 minimal media and set up to a final volume of 200 μl in clear flat bottom 96-well plates containing halflog serial dilutions of each tested compound (1 nM-80 μM) as well as high and low controls (0.2% DMSO and 10 μg/ml of norfloxacin, respectively). Upon mixing of the bacterial culture with the compounds, the OD600 of the plates was read using the Envision to account for background absorbance. The plates were then incubated in a 37° C. stationary incubator for 16 hours before measuring their OD600.

For each type of media, the duplicate EC50 measurements were analyzed independently. Bacterial growth (G) was first calculated as follows:

G=OD₆₀₀₍ₜ₌₁₆₎−OD₆₀₀₍ₜ₌₀₎

where OD600 (t=0) and OD600 (t=16) correspond to absorbance of the samples before and after incubation of the assay plates, respectively. Converting bacterial growth (G) to % residual growth (% G) was calculated as follows:

$$\% G = ( \frac{{G}_{s} - μ}{{μ}_{+} - {μ}_{-}} ) \times 100$$

where Gₛ is the bacterial growth in the presence of the tested compound, and μ+ and μ− are the averages of the high and low controls, respectively. % G was plotted against compound concentration on a semi-logarithmic plot and fit to the background corrected equation to determine EC50:

$$\% G = \frac{range}{1 + {( \frac{[ I ]}{{EC}_{50}} )}^{s}}$$

where range is the fitted % G in the absence of tested compound (inhibitor), [I] is the concentration of the tested compound (μM), S is the slope (or Hill) factor and EC50 is the compound concentration that inhibits growth by 50%.

### Determinations of Minimum Inhibitory Concentration (MIC)

Determinations of minimum inhibitory concentrations (MIC) were made for all of the compounds prioritized for follow up studies. All of these compounds were reordered from commercial suppliers. The MIC values were determined in liquid minimal media and minimal media supplemented with amino acids, vitamins and nucleobases.

A single colony of E. coli MG1655 was grown overnight in 5 ml of M9 minimal media. The saturated culture was diluted 1/50 in fresh minimal media and allowed to grow until the OD600 reached ˜0.4. The subculture was then diluted 103-fold into either fresh M9 minimal media or supplemented M9 minimal media and set up to a final volume of 200 μl in clear flat bottom 96-well plates containing 2-fold serial dilutions of each tested compound (0.25-250 μg/ml). After mixing of the bacterial culture with the compounds, the OD600 of the plates was read using Envision to account for background absorbance. The plates were then incubated in a 37° C. stationary incubator for 16 hours before measuring their OD600. After subtracting any background absorbance contributed by colored or precipitated compounds, the MIC was defined as the lowest concentration of antibiotic that inhibits visible growth.

### Metabolic Suppression Profiling

A single colony of E. coli MG1655 was grown overnight in 5 ml of M9 minimal media. The saturated culture was diluted 1/50 in fresh minimal media and allowed to grow until the OD600 reached ˜0.4. The subculture was then diluted 103-fold into fresh M9 minimal media set up to a final volume of 200 μl in clear flat bottom 96-well plates containing 4× the MIC (minimum inhibitory concentration) of each compound and a 1/20 dilution of the ChemArray stock plate. After mixing, the OD600 of the plates was read using Envision to account for background absorbance. The arrays were then incubated at 37° C. for 16 hours and their absorbance measured at 600 nm (NOTE: in the presence of selected metabolites?) Bacterial growth (G) was first calculated as above and % residual growth (% G) was calculated as follows:

$$\% G = ( \frac{{G}_{s} - {G}_{M 9}}{{G}_{M 9 ALL} - {G}_{M 9}} ) \times 100$$

where Gₛ is the bacterial growth in the presence of the tested metabolite(s), and G_{M9ALL} and G_{M9} represent the bacterial growth in minimal and supplemented minimal media, respectively.
Cloning, Expression and Purification of Recombinant PabA, PabB and PabC in E. coli.

To isolate PabA, PabB and PabC recombinant proteins, constructs were created to overexpress each protein with a N-terminal poly-histidine tag. Briefly, the genes encoding pabA, pabB and pabC were amplified from E. coli MG1655 genomic DNA using Phusion polymerase (Fermentas) using the following primers:

for pabA:
(SEQ ID NO: 1)
5′-GGGGACAAGTTTGTACAAAAAAGCAGGCTTCGAAGGAGATACTAGC
TAGATGATCCTGCTTATAGATAAC-3′;
(SEQ ID NO: 2)
5′GGGGACCACTTTGTACAAGAAAGCTGGGTCTCAGCGATGCAGGAAAT
TAGC-3′;
for pabB:
(SEQ ID NO: 3)
5′-GGGGACAAGTTTGTACAAAAAAGCAGGCTTCGAAGGAGATACTAGC
TAGATGAAGACGTTATCTCCCGCT-3′
and
(SEQ ID NO: 4)
5′-GGGGACCACTTTGTACAAGAAAGCTGGGTCTTACTTCTCCAG
TTGCTTCAG-3′;
for pabC:
(SEQ ID NO: 5)
5′-GGGGACAAGTTTGTACAAAAAA GCAGGCTTCGAAGGAGATACTAG
CTAGATGTTCTTAATTAACGGTCAT-3′
and
(SEQ ID NO: 6)
5′GGGGACCACTTTGTACAAGAAAGCTGGGTCCTAATTCGGGCG
CTCACAAAG-3′.

The PCR products were purified and cloned into pDEST17 using the Gateway cloning and Expression Kit (Invitrogen, Canada) and the constructs confirmed by DNA sequence analysis (MOBIX, McMaster University). Each construct was transformed fresh into E. coli BL21AI prior to protein expression and purification. The following procedure was followed for the expression and purification of each of the three proteins. For protein expression, each clone was grown in 2 L of LB with ampicillin (100 μg/ml) at 37° C., shaking at 250 rpm until the culture reached an OD600 of 0.6. The culture was then induced with L-arabinose and grown for an additional 3 hours prior to harvesting by centrifugation at 10,000 g. The cells were washed with a 0.85% saline solution, pelleted and stored at −20° C. For protein purification, the cell pellets was thawed and resuspended in 25 mL of lysis buffer (50 mM Tris pH=7.5, 500 mM NaCl, 15 mM imidazole, 2 mM BME, 0.5 mg DNase, 0.5 mg RNase, protease inhibitor cocktail (Roche)). Cells were lysed by passage through a French press at 30,000 psi and clarified by centrifugation at 40,000 g for 1 hour. The clarified lysate was purified by nickel chelating chromatography using a 1 mL HiTrap affinity column (GE). The column was washed with buffer A (50 mM Tris pH=7.5, 500 mM NaCl, 15 mM imidazole, 2 mM BME) and eluted with a linear gradient of 15-300 mM of imidazole. Fractions were analyzed by SDS-PAGE, and those containing pure His-tagged protein were pooled and desalted through a HiPrep 26/10 desalting column (GE) against the final storage buffer (50 mM Tris pH 7.5, 10% glycerol). The concentration of purified proteins was determined by the Bradford assay (BioRad). About 20 mg were obtained for each of the three enzymes. Fractions rich in pure protein were stored in aliquots at −80° C.

### Enzyme Assays

Enzyme assays were conducted in triplicate at room temperature with 25 nM of PabA and PabB, 50 nM of PabC, 50 mM Tris-HCl (pH 7.5), 20 μM PLP, 1 mM L-glutamine, 40 μM chorismate and the indicated concentrations of MAC-173979. The inhibition assays were initiated by addition of a mixture of the three enzymes and quenched with an equal volume of freshly prepared 8M urea. The reaction progress curves were monitored every 10 minutes for 60 minutes and determined by a stopped HPLC-assay that allowed for the quantification of the conversion of chorismate to PABA. The two compounds were separated on a C18 reverse phase column (Nova-Pak C18, 4 μm, 3.9×150 mm, Waters) and eluted isocratically with 5% acetic acid in double distilled H₂O. The analytes were visualized by UV absorbance at 275 nm and identified by comparing their retention times and UV absorption spectra to authentic standards. The progress curves were plotted to the rate equation of slow-binding inhibition:

$$[ Product ] = {v}_{s} \cdot t + \frac{( {v}_{0} - {v}_{s} )}{{k}_{obs}} \cdot ( 1 - {e}^{- {k}_{obs} \cdot t} )$$

using Sigma Plot 12.0 (SPSS, Inc., Chicago, Ill.), where v₀ and vₛ are the initial and final steady-state reaction velocities, respectively, t is the time and k_{obs} is the apparent first order rate constant for the interconversion between the initial and steady-state rates.

### Creation of Double and Triple Deletion Mutants in Threonine Catabolism

Chromosomal DNA was prepared from single deletion mutants in tdh, kbl and ltaE obtained from the Keio library (Baba et al. Mol Syst Biol 2, 2006 0008 (2006). Primers designed to amplify 500 bp upstream and downstream the deletion region in each deletion strain were as follows: for the Δtdh region: 5′-ATATTATCACCGGTACGCTTGG-3′ (SEQ ID NO: 7) and 5′-ATTTGCCCGTTGCCACTTCAATCC-3′ (SEQ ID NO: 8); for the ΔltaE region: 5′-AGGCGACAGAGCCAGAACGT-3′ (SEQ ID NO: 9) and 5′-AGACCATATCGCGCATGACTTCG-3′ (SEQ ID NO: 10) and for the Δkbl region: 5′-GAAAGAATTCTATAAATTAG-3′ (SEQ ID NO: 11) and 5′-CCCACCAGATCAAACGACAG-3′ (SEQ ID NO: 12). To create a tdh ltaE double deletion mutant, the FRT-flanked kanamycin resistance cassette in Δtdh was eliminated using the FLP helper plasmid pCP20 as previously described (Baba et al. Methods Mol Biol 416, 171-81 (2008). About 2-4 μg of purified PCR product from the ΔltaE region was transformed into the resistance marker free Δtdh strain containing pKD46 and transformants were selected on LB agar medium with kanamycin (50 μg/ml). The kanamycin resistance cassette was then eliminated from the tdh ltaE double deletion mutant by the same method described above. To create a tdh ltaE kbl triple deletion mutant, about 2-4 μg of purified PCR product from the Δkbl region was transformed into the resistance marker free Δtdh ΔltaE strain containing pKD46 and transformants were selected on LB agar medium with kanamycin (50 μg/ml). All deletion mutants were verified by PCR to confirm that the genes of interest were deleted.

### Results

### Screening for Growth Inhibitory Small Molecules in Nutrient-Deficient Media

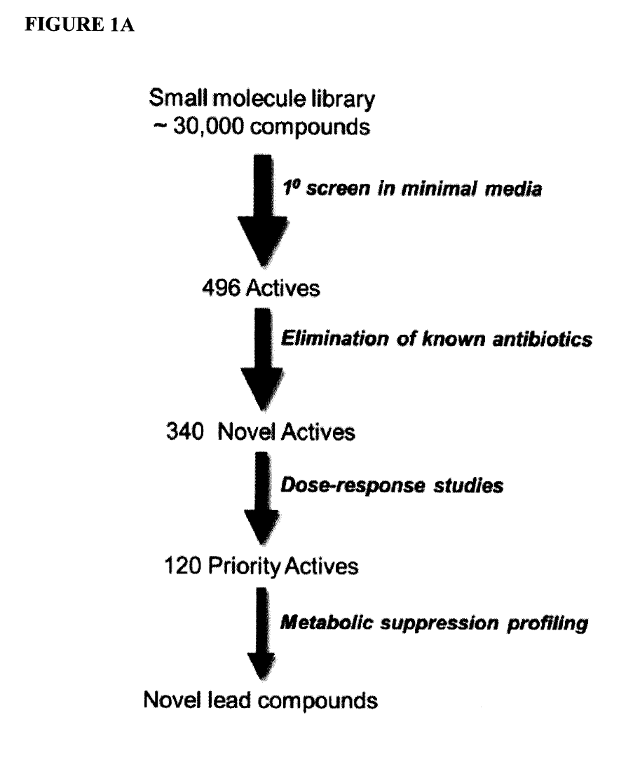

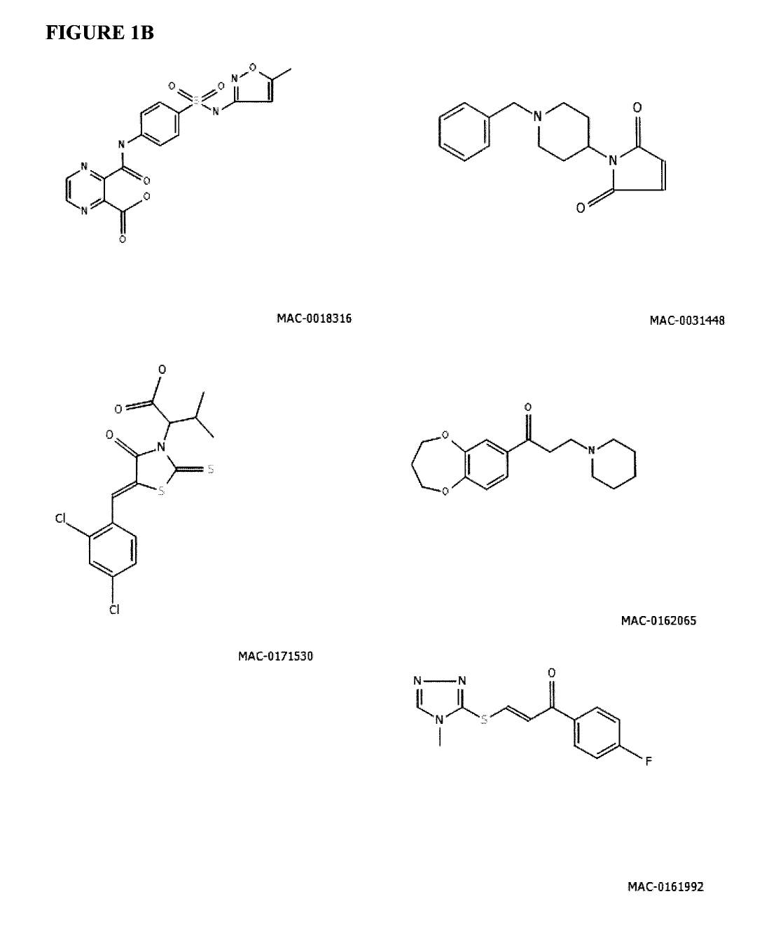

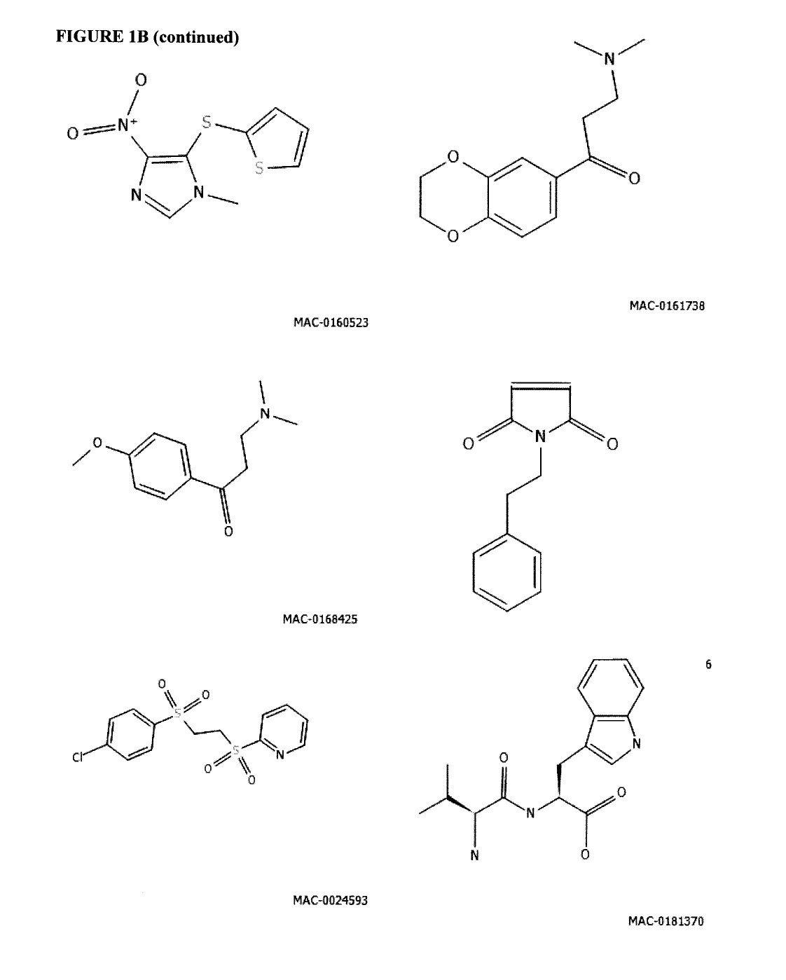

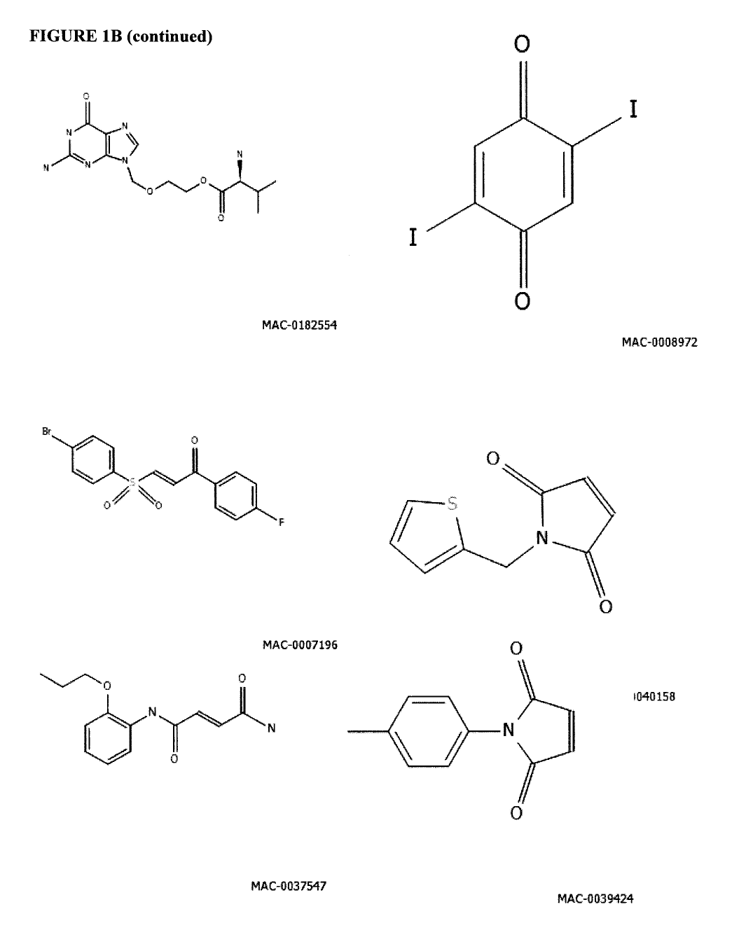

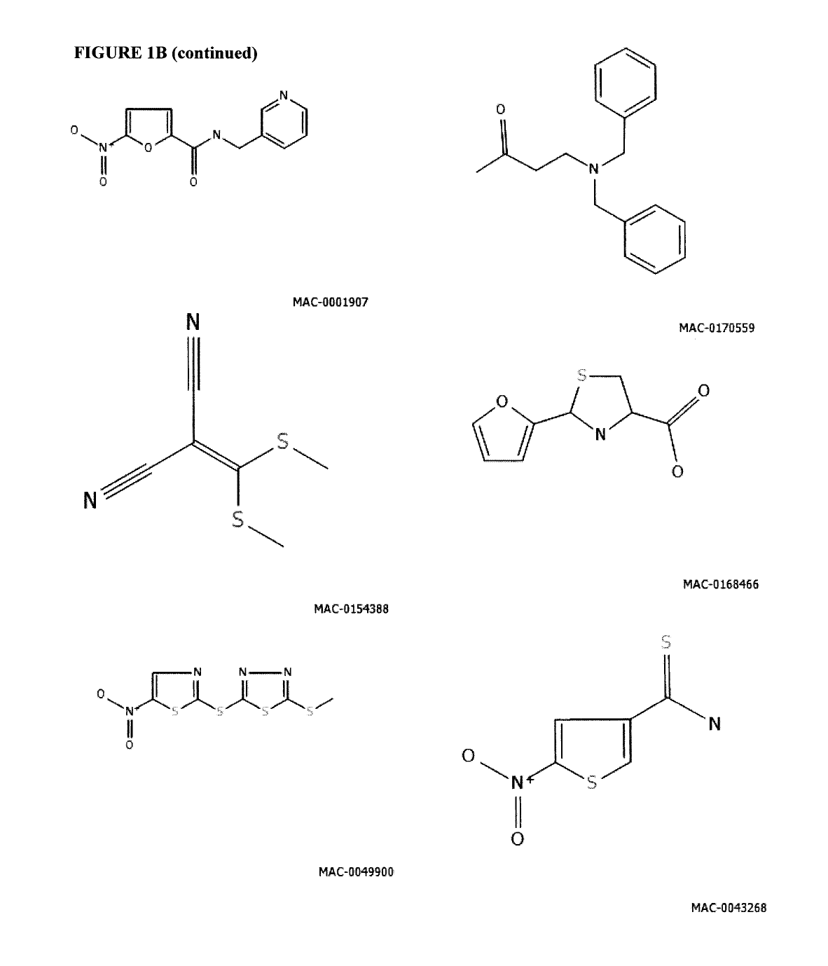

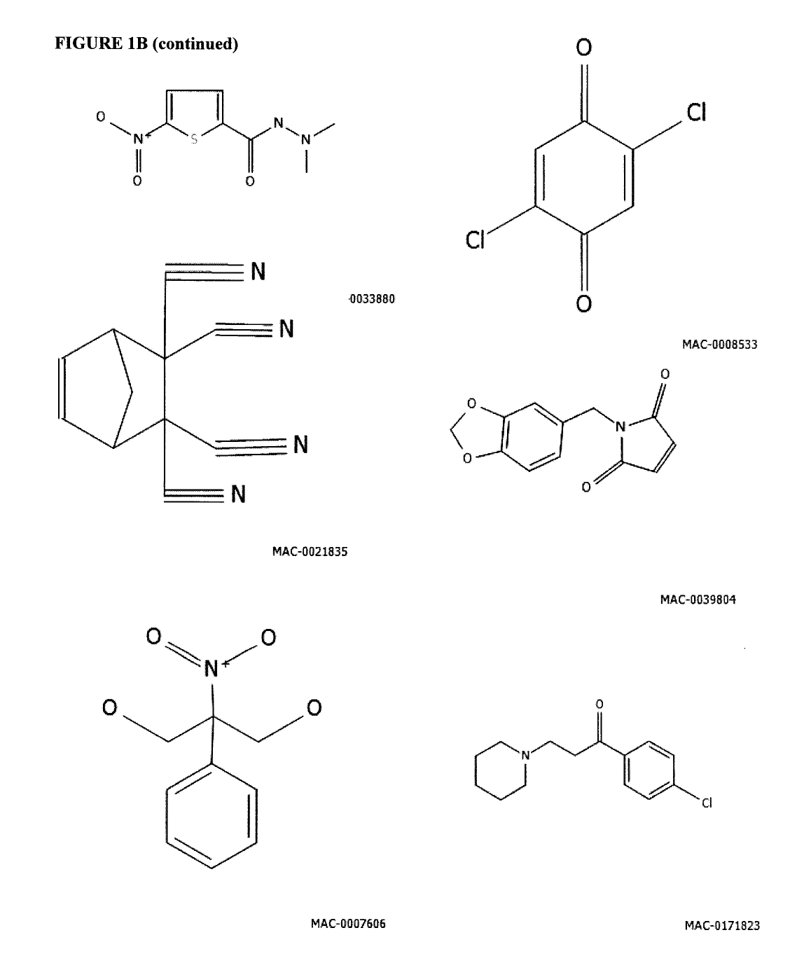

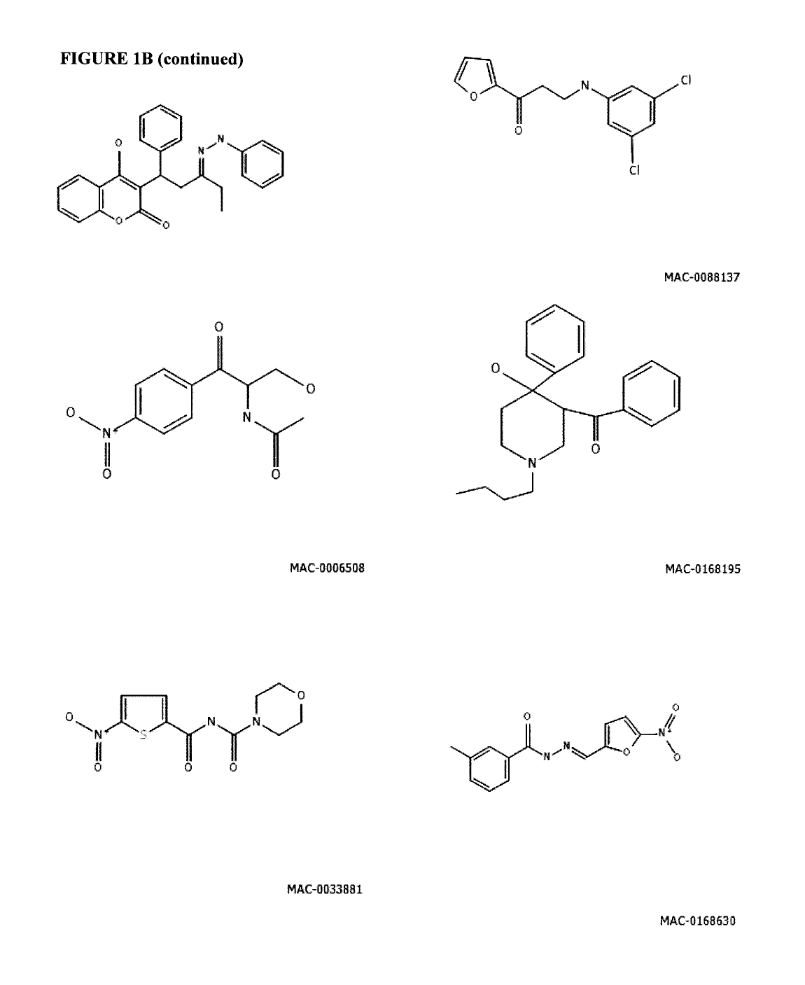

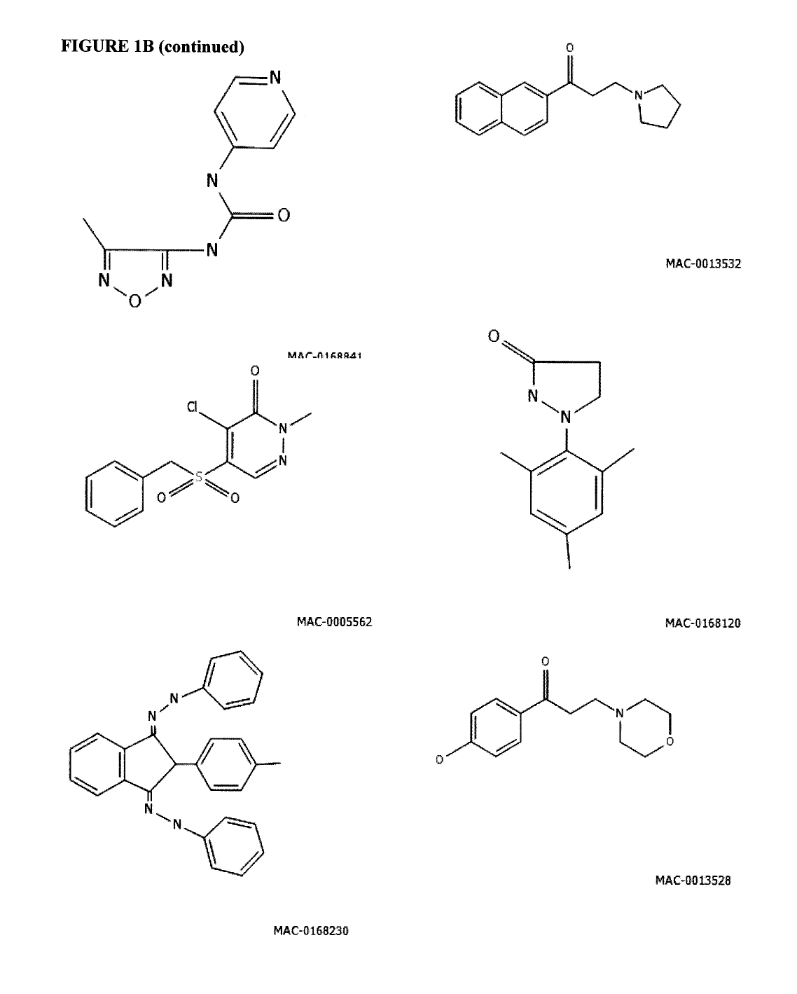

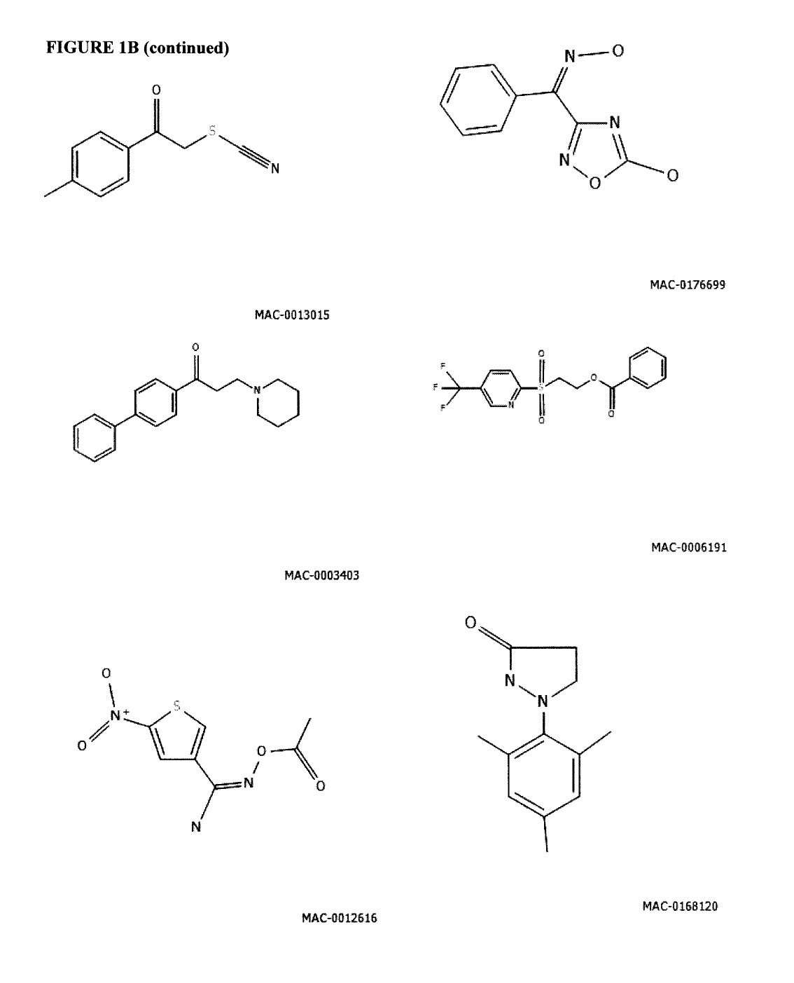

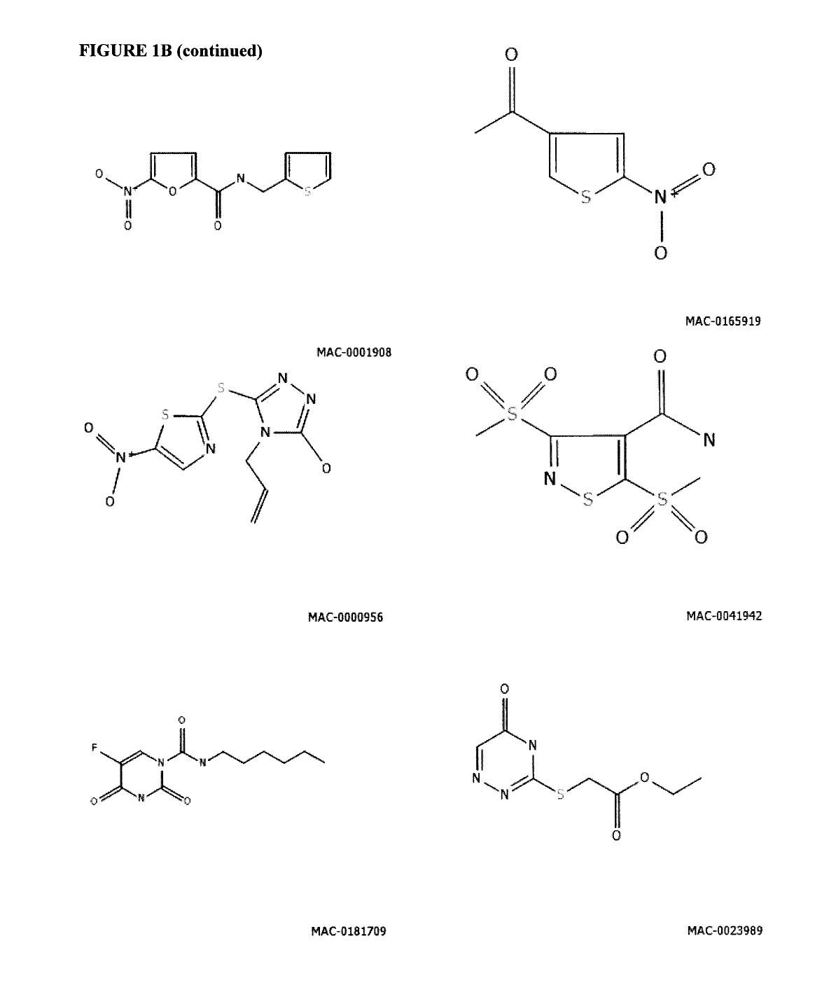

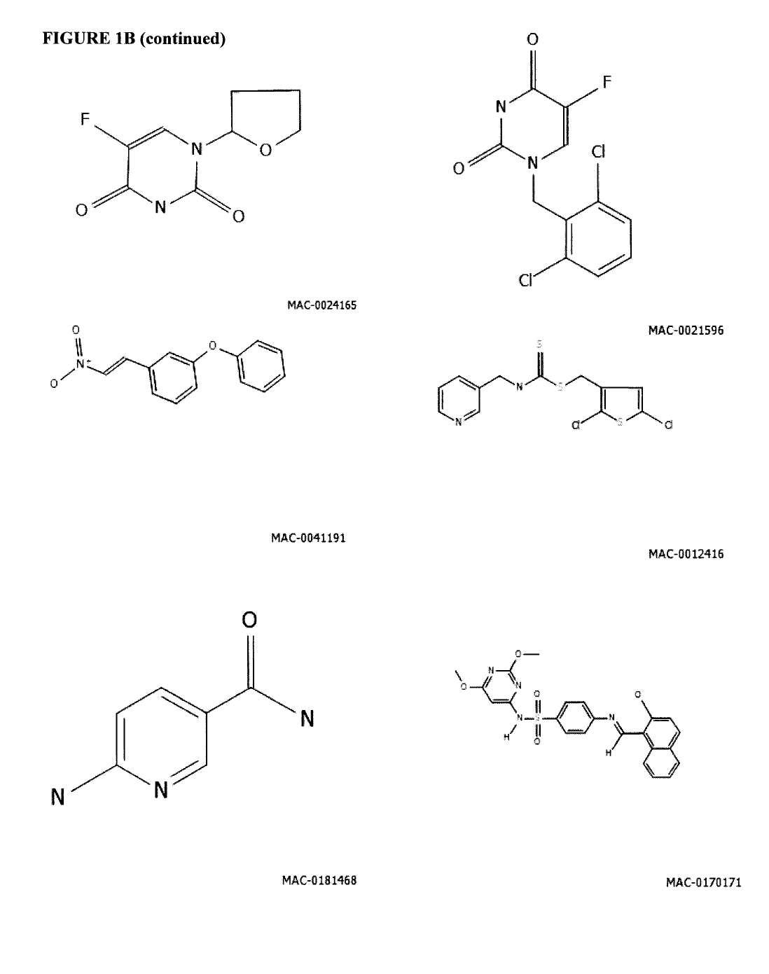

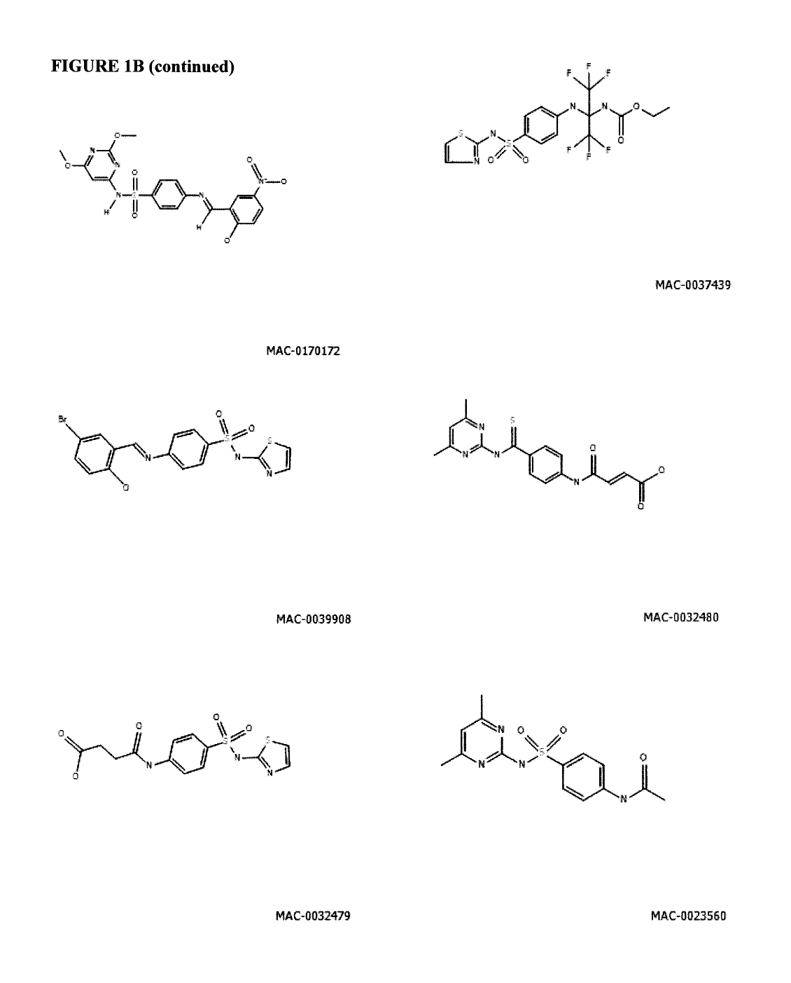

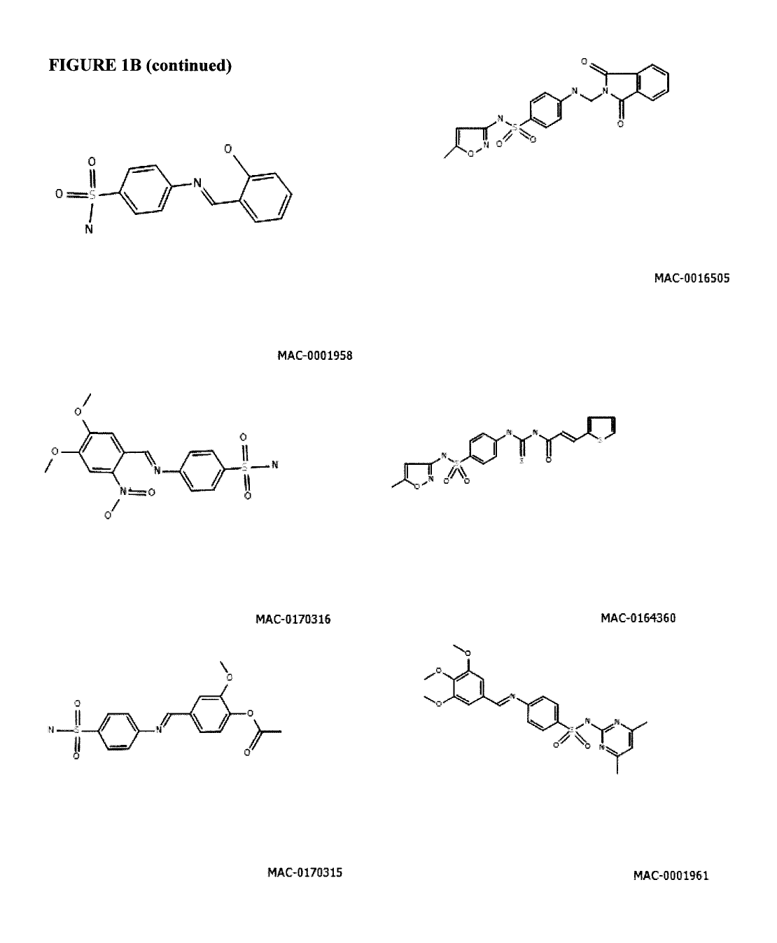

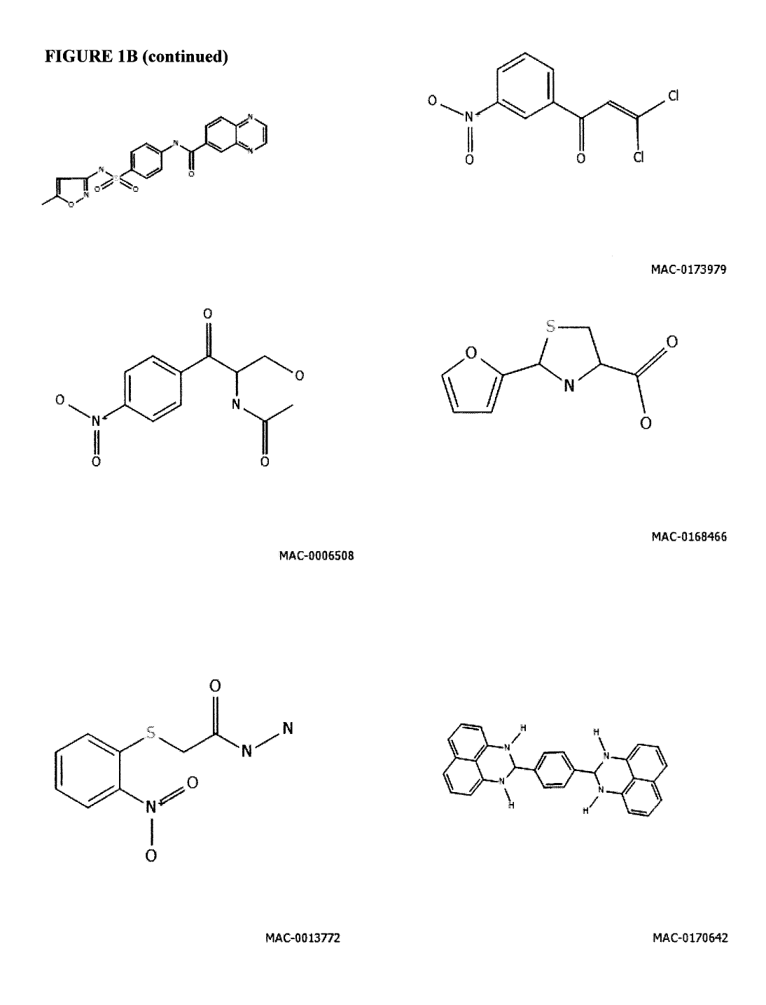

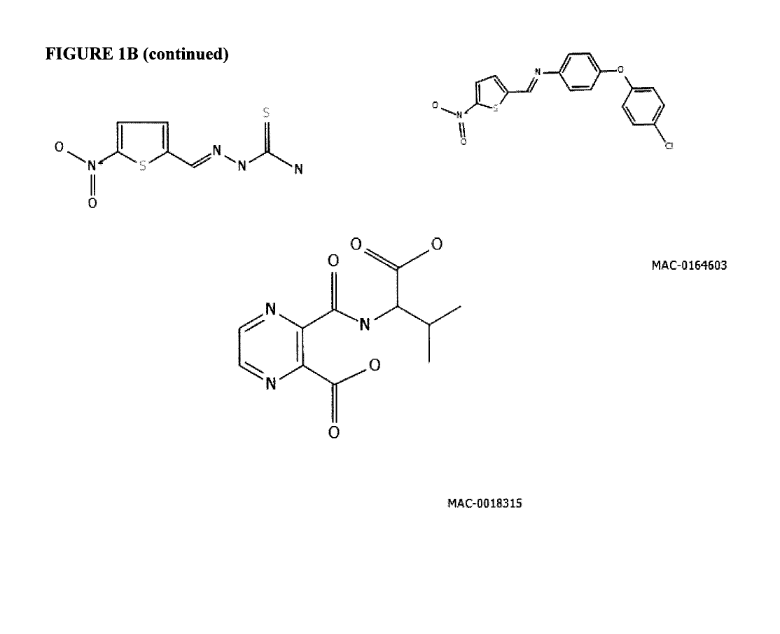

A flow chart that outlines the different stages of present work is shown in FIG. 1. The work began with a high-throughput screen to identify compounds with growth inhibitory activity at a concentration of 10 μM against E. coli MG1655 in nutrient deficient media from a library of ˜30,000 small molecules. This library, the Canadian Chemical Collection (CCC), is made up of ˜30,000 structurally diverse compounds and includes small synthetic molecules, off-patent FDA-approved and pharmacologically active molecules as well as purified natural products.

The primary screen was of high quality with respect to signal, noise and reproducibility and the compound data. The statistical parameter, Z′. describes the window between high and low controls and provides a measure to evaluate the quality of the screen. For this screen, the average Z′ value was 0.8. The hit cutoff of 80% residual growth was determined by calculating 3 standard deviations away from the high controls below 100% residual growth. This cutoff identified 496 hits that resulted in at least 20% growth inhibition relative to the high controls, corresponding to a hit rate of 1.7%. Known bioactives were eliminated from the list of hits selected for follow up resulting in a set of 340 compounds. These mainly include synthetic small molecules (MayBridge and Chembridge) since they constitute a set of novel chemical scaffolds with mostly uncharted biological activity in addition to a small number of natural products.

### Differential Media Screening to Identify Activity Specific to Nutrient-Limited Conditions

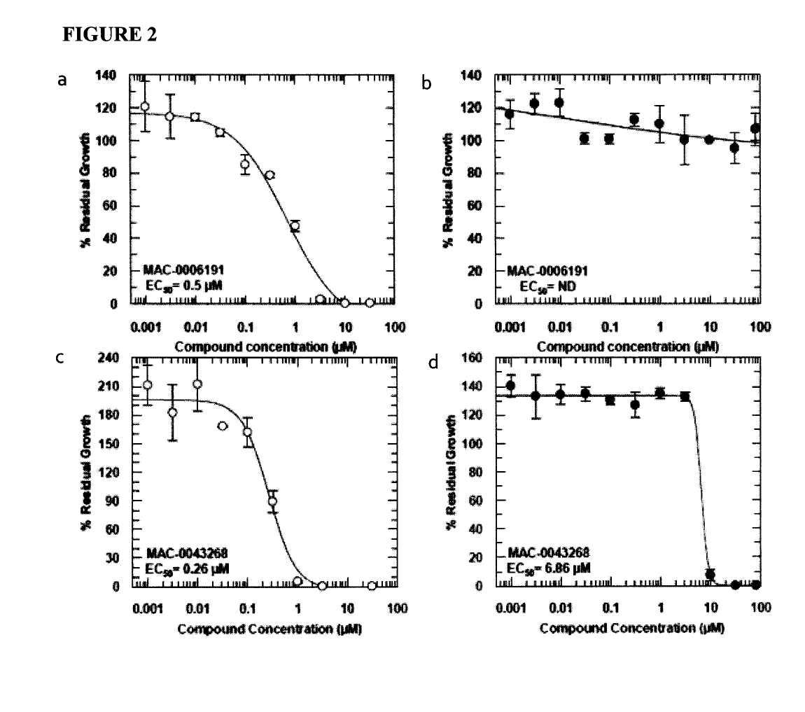

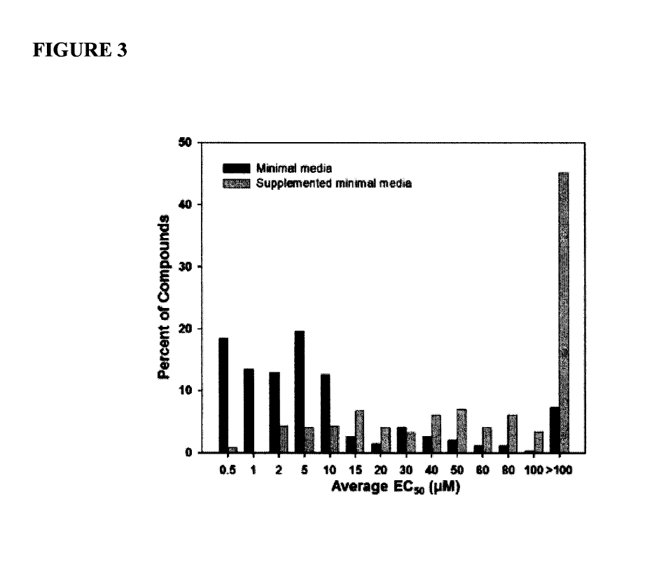

The dose-response relationship of the 340 compounds selected from the primary screen was evaluated as a first step towards characterizing their biological activity. In an effort to prioritize compounds that were specifically active under nutrient limited conditions, dose-response evaluations were conducted in nutrient-limited and in defined rich media. The differences in the EC50 values between the two conditions were considered to be indicative of the specificity of the biological activity of the small molecules to bacterial physiology under nutrient limited conditions. For all the 340 compounds, the 11-point dose response tests were carried out in duplicate in minimal media and in minimal media supplemented with a mix of amino acids, purines, pyrimidines and vitamins. The data obtained from these studies were analyzed and used to prioritize a subset of bioactives that could be explored as potential probes of bacterial physiology under nutrient limited conditions. In this respect, the dose-response relationships for each compound were evaluated based on the quality and reproducibility of the dose-response curves in nutrient limited and defined rich media in addition to the shift in EC50 values incurred by the presence of supplements in the growth media. Specifically, the fold suppression in small molecule lethality was defined as its EC50 in defined rich media divided by that in nutrient limited media. The higher the fold suppression for an inhibitor, the greater the likelihood that it targets aspects of physiology relevant to nutritionally stressed bacteria. FIG. 2 shows examples of the dose-response assessments for two compounds from the screen. The growth inhibitory activity of MAC-0006191 against E. coli MG1655 (FIG. 2a) is completely abolished when tested in defined rich media (FIG. 2b). In the case of MAC-0043268 (FIG. 2c), its EC50 increases by as much as 26-fold when evaluated in the presence of supplements (FIG. 2d). Interestingly, a large number of the 340 tested compounds exhibited a significant difference in their biological activity against E. coli between the two different growth media. In fact, as many as 45% of the compounds showed no inhibition of bacterial growth within the tested range of inhibitor concentration in defined rich media in contrast to only 7% of the compounds with no activity against E. coli in minimal media (FIG. 3). Based on the specificity and potency of biological activity in nutrient limited media, a total of 120 bioactives were prioritized for follow up analysis.

### Metabolic Suppression Profiling

Chemical complementation of auxotrophic mutants has proven to be a valuable tool in uncovering details about many biochemical pathways. The strategy presented herein relies on the use of chemical perturbation using small molecules in a way that mimics genetic perturbation as observed in auxotrophic lesions. It is therefore possible to reverse the effect of a small molecule inhibitor through chemical complementation. Thus, if a given bioactive inhibits a step in the biosynthetic pathway of a cellular metabolite, it would be possible to suppress the activity of this inhibitor by supplementing the growth medium with that particular metabolite. To this end, a secondary screen was used in which metabolite chemical complementation was used as a systematic approach to identify the potential cellular pathway(s) targeted by the bioactives prioritized from the primary screen.

In this secondary screen, growth of E. coli in minimal media containing 4× the minimum inhibitory concentration (MIC) of each tested compound was examined against an array of single primary metabolites (amino acids, vitamins and nucleobases) as well as pools of these metabolites. This array was called the Chemical Suppression Array (ChemArray) as below in Table 1:

TABLE 1

| | | | | | | | | | | | |
|---|---|---|---|---|---|---|---|---|---|---|---|
| M9 | M9 ALL | AA | VIT | NUC | AA + VIT | AA + NUC | VIT + NUC | AA + VIT | AA + NUC | VIT + NUC | M9 ALL |
| D-Ala | L-Ala | Gly | Glu | Ser | Iso | Leu | Met | Trp | Arg | Cys | AA |
| His | Val | Pro | Lys | Thr | Asn | Asp | Phe | Tyr | Gln | M9 | VIT |
| A | T | G | U | Vit. B7 | Vit. B3 | Vit. B5 | Vit. B6 | Vit. B1 | Vit. B2 | PABA | NUC |

The metabolite(s) that suppress compound lethality shed light on the potential pathway(s) targeted by these bioactives.

A clustered heat map was generated to show the metabolic suppression profile of 74 prioritized bioactives and of a set of known antibiotics with different modes of action as controls to validate the approach. In this heat map, the interaction of the bioactives (y-axis) with each metabolite (x-axis) is based on how well that metabolite suppresses the inhibitory activity of the small molecule. The overall patterns of interaction between metabolites and small molecule inhibitors of nutritionally stressed bacteria create unique metabolic suppression fingerprints that can be used to guide hypotheses regarding the mechanism of action (MOA) of these inhibitors. The heat map is clustered based on these metabolic suppression fingerprints so that compounds with similar profiles are grouped within the same cluster.

### Metabolic Suppression Profiles of Known Antibiotics

The metabolic suppression profiles of 7 well-known antibiotics demonstrate the power of this approach towards exploring the MOA of inhibitors that namely target biosynthetic pathways and more generally impair bacterial physiology under nutrient-limited conditions. Noteworthy in this respect was that the activity of known antibiotics with mechanisms that do not directly involve primary metabolism such as translation, replication and transcription inhibitors (e.g. tetracycline, norfloxacin and rifampicin, respectively) is not altered in the presence of supplements. On the other hand, the activity of the cell wall inhibitor, D-cycloserine, was suppressed by the addition of either D/L-alanine or glycine to the media as well as by pools containing a mixture of amino acids. This suppression pattern can be explained both at the level of transport and of MOA of D-cycloserine. D-cycloserine is known to use the same import mechanism employed by D-alanine and glycine and encoded by the transporter cycA. Addition of D-alanine or glycine to the growth media antagonizes the action of D-cycloserine by preventing its entry into the cell. Interestingly, the ChemArray contains a mixture of both the D- and L-isomers of alanine and each isomer on its own fully suppressed growth inhibition by D-cycloserine (data not shown). Inside the cell, D-cycloserine targets two processes. It acts as a competitive inhibitor of the enzyme, D-ala-D-ala-ligase (Ddl) in peptidoglycan biosynthesis. It also competitively inhibits a second enzyme encoded by dadX, D-alanine racemase, which catalyzes the interconversion of D- and L-alanine. It can therefore be understood how addition of L-alanine to the growth media would outcompete D-cycloserine in binding to DadX and result in an increase in the D-alanine pool inside the cell through the DadX-catalyzed racemation. D-alanine would in turn prevent the binding of D-cycloserine to Ddl thus overcoming its growth inhibitory effect.

The inhibitory activity of the anti-folate antibiotic, sulfamethoxazole, was shown to be fully reversed in the presence of p-aminobenzoic acid (PABA) and to a certain extent in the presence of methionine as well as metabolic pools containing all amino acids and/or all vitamins. This pattern of chemical complementation is a signature of inhibitors of PABA metabolism. The enzymes, PabA, PabB and PabC, catalyze the biosynthesis of PABA from chorismate. PABA then serves as a precursor of the essential folate coenzymes which are involved in the transfer of one-carbon units in several cellular pathways including the biosynthesis of methionine, purines and pyrimidines. Sulfamethoxazole and other sulfa drug inhibitors exert their antibacterial activity by competing with PABA at the step of dihydropteroate synthesis (catalyzed by FolP) and blocking its entry into the biosynthetic pathway of tetrahydrofolate. They are also incorporated into the pathway as alternate substrates ultimately creating dead-end products that cannot serve as substrates for the synthesis of dihydrofolate. The addition of PABA to the growth media outcompetes sulfamethoxazole, enabling the cells to overcome growth inhibition. Methionine is one of the major cellular metabolites that require folate cofactors for their biosynthesis. Adding it to the growth medium can partially reduce the cellular requirement for folates and alleviates inhibition by sulfa drugs.

Trimethoprim is an antibiotic that targets dihydrofolate reductase, encoded by folA, which catalyzes the synthesis of tetrahydrofolate. Given that derivatives of this coenzyme are essential for the transfer of one-carbon units in many cellular processes including the biosynthesis of glycine, methionine, pantothenate, formylated methionine as well as purine and pyrimidine nucleotides, growth inhibition by trimethoprim was only be suppressed by providing a mixture of all supplements or at least a mixture of amino acids and nucleobases.

The herbicide glyphosate inhibits the product of aroA, 5-enol-pyruvylshikimate-3-phosphate synthase is involved in the biosynthesis of chorismate which in turn serves as a precursor of several metabolites, most importantly the aromatic amino acids, phenylalanine, tyrosine and tryptophan. Interestingly, suppression of the antibacterial activity of glyphosate could only be achieved by providing a mixture of amino acids in the growth media. This is expected since by inhibiting chorismate biosynthesis, glyphosate creates multiple auxotrophic requirements for the three aromatic amino acids.

Metabolic suppression fingerprints similar to those of trimethoprim and glyphosate, in which growth inhibition was reversed by the complete pool of metabolites or amino acids, were observed for almost 25% of the priority bioactives profiled by the ChemArray. This observation revealed the need to enrich the ChemArray with additional pools of metabolites that could shed more light on bioactives that would target early steps in branched metabolic pathways and would require more than one supplement to suppress their activity. In principle, the number of possible combinations of metabolites is very large. For practical considerations, a survey of primary metabolism in E. coli unveiled a number of pathway intermediates and metabolite pools that were added to create the expanded chemical suppression array as shown in Table 2:

TABLE 2

| | | | | | | | | | | |
|---|---|---|---|---|---|---|---|---|---|---|
| M9 | Ala | Gly | Glu | Ser | Iso | Vit. B7 | DHQ | Thr-Met | Gly-Ile | M9 |
| | | | | | | | | Lys | | |
| M9 ALL | Leu | Met | Trp | Arg | Cys | Vit. B3 | SHIK | Thr-Met | Leu-Val | M9 ALL |
| | | | | | | | | Ile | Ala-B5 | |
| AA | His | Val | Pro | Lys | Thr | Vit. B5 | 4-HBA | Thr-Met | 5-ALA | AA |
| | | | | | | | | DAP-Lys | | |
| VIT | Asn | Asp | Phe | Tyr | Gln | Vit. B6 | 2,3-DHB | Lys-Met | Gly-Met | VIT |
| | | | | | | | | | B5-T-A | |
| NUC | A | T | G | U | Trace | Vit. B1 | Tyr-Phe | Met-Thr | Homoser | NUC |
| | | | | | Metals | | | | | |
| AA + VIT | PUR | PYR | PUR | NUC | Iron | Vit. B2 | Aro AA | Met-Ile | CIT | AA + VIT |
| | | | His | His | | | | | | |
| AA + NUC | Pool 1 | Pool 2 | Pool 3 | Pool 4 | Pool 5 | PABA | Aro AA | Ile-Val | ORN | AA + NUC |
| | | | | | | | PABA | | | |
| VIT + NUC | Pool 6 | Pool 7 | Pool 8 | Pool 9 | Pool 10 | Vit. B12 | Aro | Thr-Met | PUT | VIT + NUC |
| | | | | | | | | Lys-Leu | | |
| | | | | | | | | Ile-Val | | |

PUR: purine nucleobases;
PYR: pyrimidine nucleobases;
DHQ: dihydroquinone;
SHIK: shikimic acid;
4-HBA: 4-hydroxybenzoic acid;
2,3-DHB: 2,3-dihydroxybenzoic acid;
Aro AA: aromatic amino acids (Phe, Tyr, Trp);
Aro: aromatic amino acids,
p-aminobenzoic acid, 4-hydroxybenzoic acid and 2,3-dihydroxybenzoic acid;
DAP: diaminopimelic acid;
5-ALA: 5-aminlevulinic acid;
Homoser: homoserine;
CIT: citrulline;
ORN: ornithine; PUT: putrescine.

Most of these involve highly regulated and entwined pathways such as the biosynthesis of aromatic or branched-chain amino acids. Thus, in contrast to single supplements, pools of metabolites containing a mixture of the aromatic amino acids in the expanded ChemArray fully reversed the growth inhibitory activity of glyphosate. This reveals how carefully chosen mixtures of metabolites could provide information directly pertaining to the MOA of a small molecule inhibitor. After profiling them through the expanded ChemArray, the activity of over half of the compounds that were not suppressed by single supplements was antagonized by different pools of metabolites in the array.

MAC-0173979 Inhibits p-Aminobenzoic Acid Biosynthesis in E. coli.

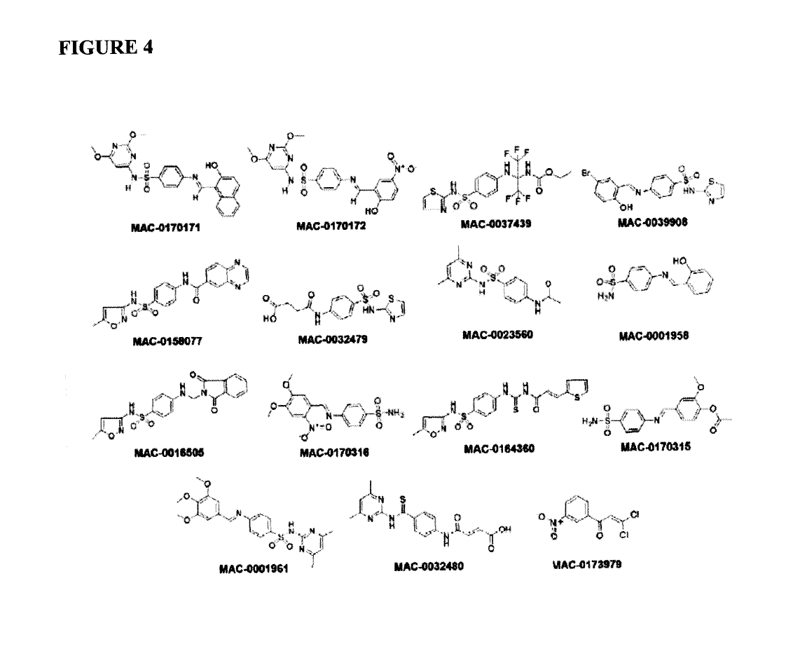

One of the major clusters revealed in the generated heat map grouped the metabolic suppression profiles of 16 bioactives including that of sulfamethoxazole, namely MAC-0170171, MAC 0170172, MAC-0037439, MAC-0039908, MAC-0032480, MAC-0032479, MAC-0023560, MAC-00011958, MAC-0016505, MAC-0170316, MAC-0164360, MAC-0170315, MAC-0001961, MAC-0158077 and MAC-0173979. Like the sulfa drug, these compounds were suppressed when PABA, or to a certain extent methionine, were present in the growth media. As mentioned earlier, this fingerprint of metabolic complementation is a trademark of inhibitors of PABA metabolism. When looking at the structures of the molecules in this cluster (FIG. 4), it can be seen that the majority are structural analogs of sulfa drugs validating the present strategy.

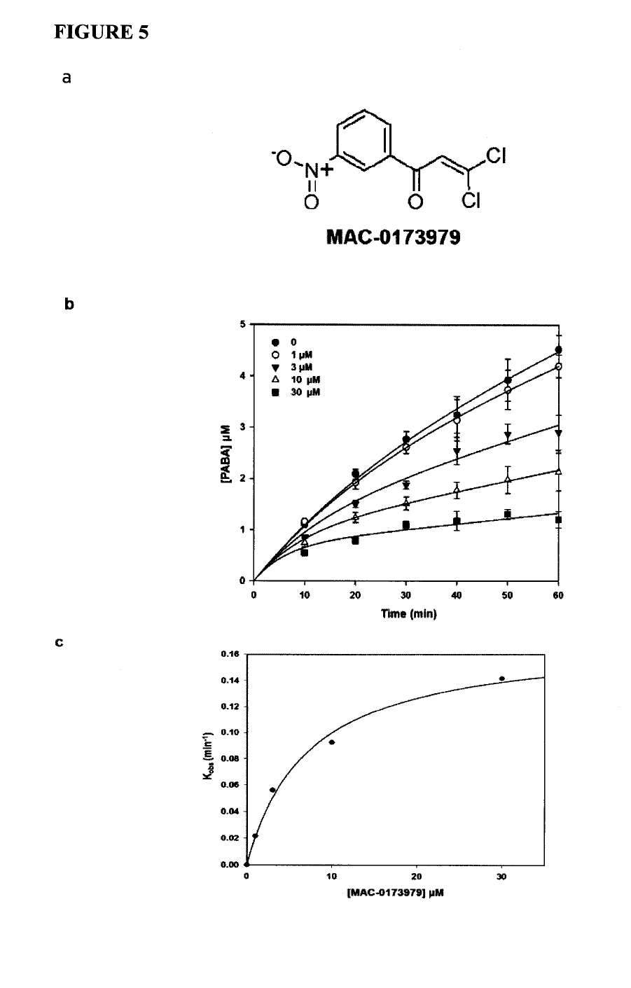

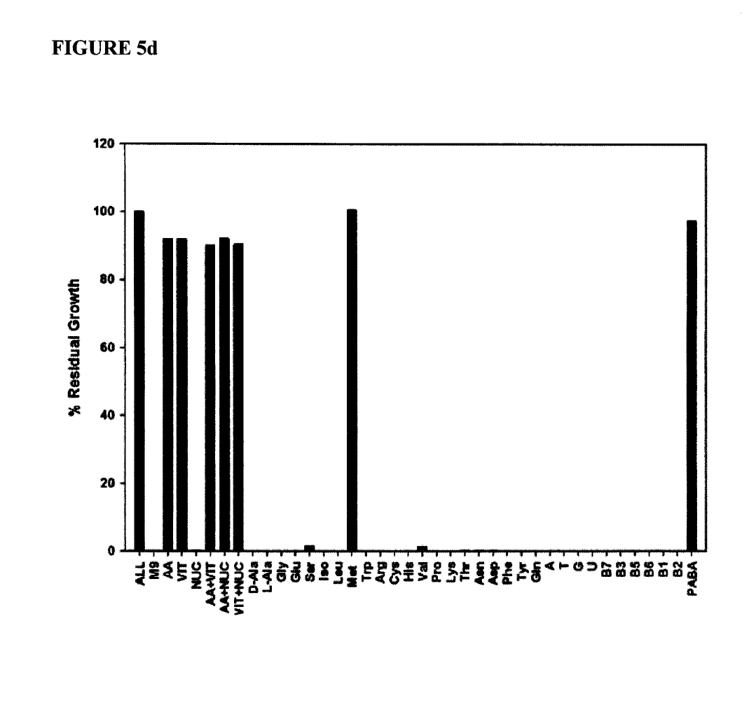

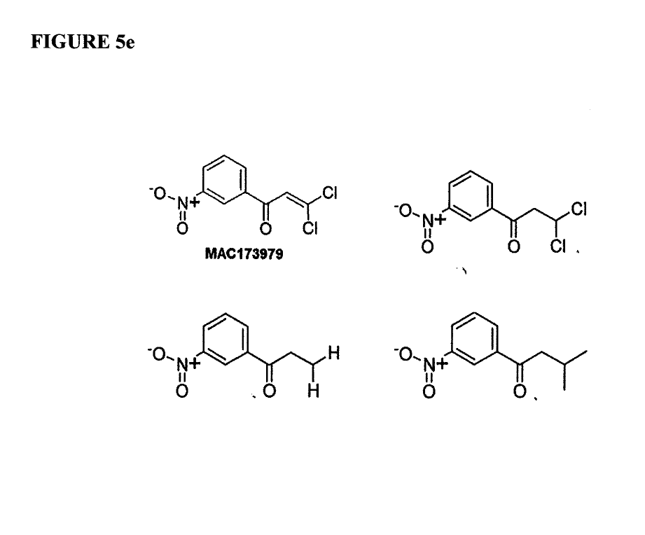

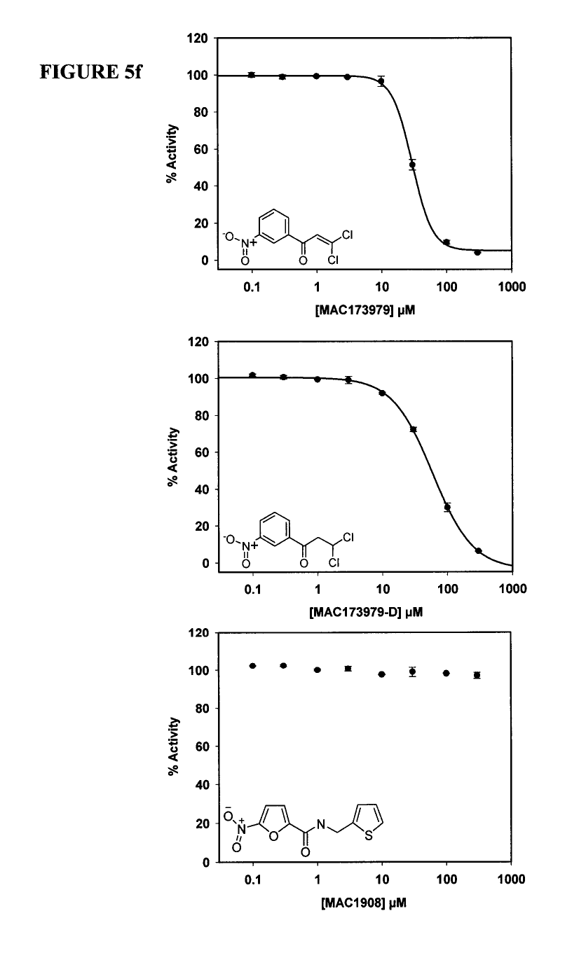

The exception to this trend is the inhibitor MAC-0173979 which is a dichloro-nitrophenyl propenone which is not structurally similar to sulfonamides (FIG. 5a). Both PABA and methionine fully reversed the activity of MAC-0173979 (FIG. 5d). Furthermore, the addition of PABA to the growth media resulted in a 16-fold suppression of its MIC. This effect was not observed when other metabolites derived from chorismate were tested. Given its specific metabolic suppression profile and its unique structure suggesting an inhibition mechanism different from that of sulfa drugs, MAC-0173979 was tested for inhibition of the folate pathway at the branch of PABA biosynthesis. PABA is synthesized from chorismate and L-glutamine in two steps catalyzed by three enzymes, PabA, PabB and PabC. PabA and PabB form a heterodimer in which PabA acts as a glutaminase, releasing free ammonia from L-glutamine to be used by PabB to aminate chorismate to form 4-amino-4-deoxychorismate (ADC). PabC, ADC lyase, aromatizes ADC forming PABA and releasing pyruvate.

In order to directly test the biochemical activity of MAC-0173979 on PABA synthesis, an HPLC-based one-pot enzyme assay using recombinant PabA, PabB and PabC was set up and the formation of PABA was monitored by measuring its absorbance at 275 nm. On addition of an enzyme mixture of PabA, PabB and PabC to initiate the synthesis of PABA from chorismate and L-glutamine in the presence of different concentrations of MAC-0173979, the resulting reaction progress curves followed a curvilinear trend whereby each curve reached a slower steady-state rate after a fast initial velocity (FIG. 5b). This is characteristic of time-dependent enzyme inhibition.

FIG. 5c shows that the plot of the k_{obs} values derived from the progress curves in FIG. 5b versus [MAC-0173979] fits a hyperbolic function consistent with a mechanism of time-dependent inhibition that involves an isomerization of the EI complex suggesting that MAC-0173979 is an irreversible time-dependent inhibitor of PABA synthesis with an apparent Ki of 7.3±1.3 μM.

MAC173979 and an analog lacking the Michael acceptor (0-1000 μM) were each incubated with the PABA enzymes for 30 minutes prior to substrate addition. Reactions were quenched after 30 minutes with 8 M urea and analyzed by HPLC. As a negative control, an inhibitor not suppressed by PABA, MAC1908, was also tested. Data with the error bars represent the mean % activity±range of n=2 replicates and the dose response curves were fitted to the four parameter logistic nonlinear regression curve yielding IC₅₀ values of 30±2 μM for MAC173979 and 60±7 μM for its analogue and no inhibition for the negative control.

MAC-0168425 and Analogues Interfer with Glycine Metabolism in E. coli

A second cluster in the generated heat map shows the metabolic suppression profiles of 8 bioactives including that of D-cycloserine, namely MAC-0161992, MAC-0162065, MAC-0160523, MAC-0161738, MAC-0168425, MAC-0031946 and MAC-0024593. While no two molecules in this cluster have the same metabolic suppression fingerprint, they are all strongly suppressed by the amino acid, glycine. The profile of D-cycloserine has been discussed above.

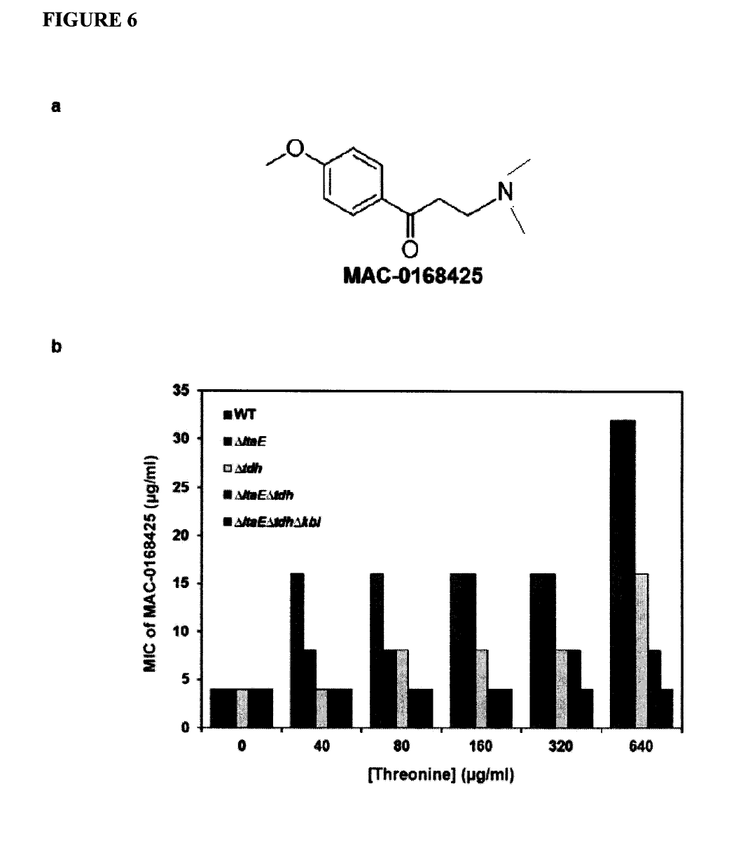

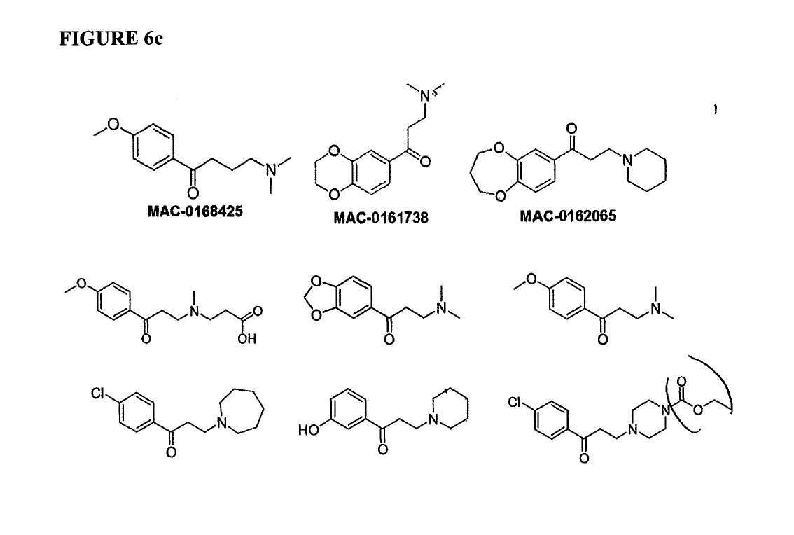

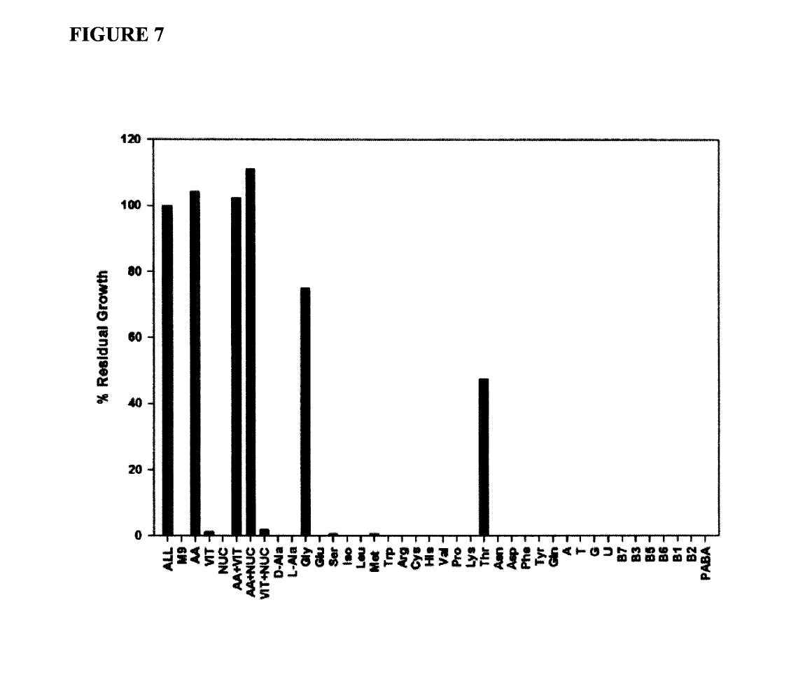

The activity of MAC-0168425 (FIG. 6a) is strongly suppressed by glycine and to a lesser extent by L-threonine (FIG. 7). In E. coli, glycine is primarily synthesized from serine in a one-step reaction catalyzed by serine-hydroxymethyl transferase (product of glyA). Serine is in turn synthesized from the glycolytic intermediate, 3-phosphoglycerate, through the action of three enzymes encoded by serA, serC and serB. This pathway is not only a source of serine and glycine for protein synthesis but is also the major source of one-carbon units needed for other cellular pathways such as the synthesis of methionine, thymine, purines and pantothenate. Threonine catabolism also contributes to the cellular pool of glycine. In a two-step pathway, threonine dehydrogenase oxidizes threonine to α-amino-β-ketobutyrate which is then cleaved by α-amino-β-ketobutyrate lyase to form glycine and acetyl CoA. This pathway is considered the major secondary pathway for glycine production. A second minor pathway of threonine degradation is catalyzed by the activity of LtaE, low-specificity threonine aldolase to form glycine and acetaldehyde. Given that MAC-0168425 is strongly suppressed by glycine, partially suppressed by L-threonine and not suppressed by L-serine, the connectivity between L-threonine and glycine metabolism appears to underlie the partial suppression by L-threonine observed in the profile of MAC-0168425.

The capacity of L-threonine to suppress the activity of MAC-0168425 in strains impaired in the aforementioned threonine degradation pathways was evaluated. This was explored by looking for shifts in the MIC of MAC-0168425 in the presence of different concentrations of L-threonine in different deletion mutants in threonine catabolic pathways. In the wild-type strain, MAC-0168425 has a 4-8 fold shift in its MIC in the presence of 40-640 μg/ml of L-threonine in the media (FIG. 6b). Within this range, L-threonine is generally less effective at suppressing growth inhibition by MAC-0168425 in a Δtdh mutant than in a ΔltaE mutant (FIG. 6b). This observation is consistent with the fact that the Tdh-Kbl mediated threonine catabolic pathway plays a more significant role in replenishing the cellular pool of glycine. A deletion mutant deficient in both the major and minor pathways of threonine conversion to glycine, ΔltaEΔtdh was constructed as described previously. In this double deletion strain, the activity of MAC-0168425 is only suppressed at the highest concentrations of L-threonine tested (320-640 μg/ml) sustaining a 2-fold shift in its MIC (FIG. 6b). This phenotype may be due to a non-specific oxidation at high concentrations of L-threonine to form α-amino-β-ketobutyrate which can then be cleaved by α-amino-β-ketobutyrate lyase (Kbl) to produce glycine and partially suppress growth inhibition by MAC-0168425. In a triple deletion mutant strain (ΔltaEΔtdhΔkbl), suppression of the activity of MAC-0168425 is completely lost (FIG. 6b). It should be noted herein that the double and triple deletion mutants grow similarly to the wild-type parent strain and to the single deletion mutants (data not shown). These data indicate that the suppression of the lethality of MAC-0168425 by L-threonine is mediated through its conversion to glycine inside the cell and that this molecule elicits its growth inhibitory activity by chiefly interfering with glycine metabolism.

### Example 2

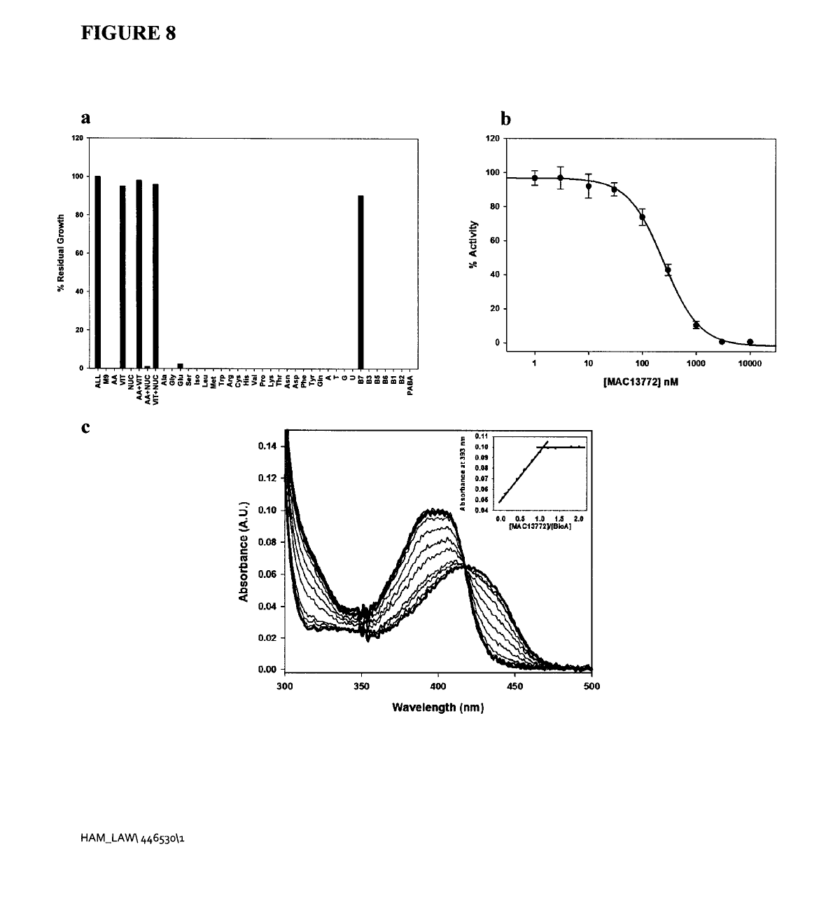

Using the methods detailed in Example 1, the compound MAC-0013772 was determined to be uniquely suppressed by biotin in the growth media (FIG. 8a). The late steps of biotin synthesis are well understood to be catalyzed by the enzymes BioF, BioA, BioD and BioB. Given that E. coli cells are permeable to the late intermediates in biotin biosynthesis, the suppression of the inhibitory activity of MAC-0013772 in the presence of 7-keto-8-aminopelargonate (KAPA), 7,8-diaminopelargonate (DAPA) and dethiobiotin (DTB) in comparison to unsupplemented and biotin controls was tested. As summarized in Table 2, inhibition by MAC-0013772 was fully reversed by DAPA, DTB and biotin, i.e. the products of BioA, BioD and BioB reactions, respectively.

TABLE 2
Antibacterial activity of MAC-0013772 against E. coli MG1655
in the presence of intermediates of biotin biosynthesis

| Supplement ^{a} | MIC (μg/ml) ^{b} | Fold suppression ^{c} |
|---|---|---|
| None | 8 | — |
| AA + VIT + NUC | >256 | 32 |
| KAPA | 8 | 1 |
| DAPA | >256 | 32 |
| DTB | >256 | 32 |
| BIOTIN | >256 | 32 |

^{a} AA: amino acids, VIT: vitamins, NUC: nucleobases, KAPA: 7-keto-8-aminopelargonate, DAPA: 7,8-diaminopelargonate and DTB: dethiobiotin
^{b} MICs were determined as described in Methods. Values are representative of three independent experiments
^{c} Fold suppression is the ratio of the MIC in the presence of the supplement to the MIC without supplementation

In contrast, KAPA had no effect on MAC-0013372 activity. To determine if the step catalyzed by BioA was the target of this inhibitor, the inhibitory activity of MAC-0013772 against recombinant E. coli BioA was assayed through a feeding assay of a bioA auxotroph. The dose-response curve shows that MAC-0013772 is a potent inhibitor of BioA with an IC₅₀ of ˜250±28 nM (FIG. 8b).

To further determine if the inhibition of BioA by MAC13772 is mediated through the interaction of the hydrazine moiety in the compound with PLP in the active site of the enzyme, the UV-visible spectra of BioA was assessed when titrated with the inhibitor. As shown in FIG. 8c, the interaction of MAC-0013772 with BioA is associated with a shift in the λₘₐₓ of the internal aldimine of the PLP-bound enzyme from 420 nm to 393 nm representing the newly formed PLP-inhibitor adduct. The molar ratio plot of [MAC-0013772]/[BioA] indicates that the interaction between the protein and the ligand is stoichiometric.

Having established the biochemical interaction of BioA with MAC-0013772, the structure-activity relationship (SAR) of this compound was studied by determining the antibacterial and biochemical activity of analogs as set out in Table 4:

TABLE 4
Structure-activity relationships of MAC-0013772 and analogs against E. coli

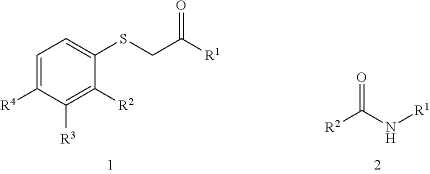

| | | | | | MIC (μg/ml)^{a} | | % Inhibition^{b} | |
|---|---|---|---|---|---|---|---|---|
| Compound | R¹ | R² | R³ | R⁴ | −bio | +bio | 1 μM | 10 μM |
| MAC13772 | NHNH₂ | NO₂ | H | H | 8 | >256 | 100 | 100 |

*Effect of Substitution on Benzene ring in Series 1

| | | | | | | | | |
|---|---|---|---|---|---|---|---|---|
| 1a | NHNH₂ | H | NO₂ | H | 256 | >256 | 42 | 84 |
| 1b | NHNH₂ | H | H | NO₂ | 64 | >256 | 66 | 100 |
| 1c | NHNH₂ | H | H | H | 64 | >256 | 49 | 100 |
| 1d | NHNH₂ | F | H | H | 32 | >256 | 36 | 100 |
| 1e | NHNH₂ | Cl | H | H | 16 | >256 | 63 | 100 |
| 1f | NHNH₂ | OH | H | H | 256 | 256 | 32 | 70 |
| 1g | NHNH₂ | NH₂ | H | H | 256 | >256 | 58 | 100 |
| 1h | NHNH₂ | CH₃ | H | H | 16 | >256 | 81 | 100 |
| 1i | NHNH₂ | OCH₃ | H | H | 128 | >256 | 65 | 100 |

*Effect of changing hydrazine functionality on R¹ in Series 1

| | | | | | | | | |
|---|---|---|---|---|---|---|---|---|
| 1j | NHNHAc | H | H | NO₂ | >256 | >256 | 0 | 0 |
| 1k | CH₂CH₃ | NO₂ | H | H | >256 | >256 | 0 | 0 |
| 1l | NH₂ | NO₂ | H | H | >256 | >256 | 0 | 0 |
| 1m | CH₃ | NO₂ | H | H | >256 | >256 | 0 | 0 |
| 1n | OH | NO₂ | H | H | >256 | >256 | 0 | 0 |

*Activity of the side chain of the parent molecule

| | | | | | | | | |
|---|---|---|---|---|---|---|---|---|
| 2a | NH₂ | CH₂SCH₃ | — | — | 64 | 64 | 50 | 64 |
| 2b | NH₂ | CH₂SH | — | — | 256 | 256 | 20 | 34 |
| 2c | NH₂ | CH₂CH₂CH₃ | — | — | >256 | >256 | 1 | 40 |
| 2d | NH₂ | CH₂CH₃ | — | — | >256 | >256 | 0 | 8 |
| 2e | NH₂ | CH₃ | — | — | 128 | 128 | 7 | 19 |

*Effect of different ring substituents in R² in Series 2^{c}

| | | | | | | | | |
|---|---|---|---|---|---|---|---|---|
| 2f | NH₂ | Benthiophene | — | — | >256 | >256 | 15 | 69 |
| 2g | NH₂ | CH₂SCH₂Ph | — | — | 64 | >256 | 60 | 100 |
| 2h | NH₂ | CH₂SNaph | — | — | >256 | >256 | 71 | 100 |
| 2i | NH₂ | CH₂SPyr | — | — | 64 | >256 | 32 | 77 |
| 2j | NH₂ | Nitrobenzyl | — | — | >256 | >256 | 0 | 29 |

^{a}MICs are determined against E. coli MG1655 in absence and presence of 2 nM of biotin
^{b}The biochemical activity of analogs is determined against recombinant E. coli BioA through a feeding assay of a bioA auxotroph at 1 and 10 μM and expressed as a % of the respective DMSO control
^{c}Abbreviations: Benthiophene: benzothiophene; Ph: phenyl; Naph: naphthalenyl; Pyr: pyridine

TABLE 5
Structures of MAC-0013772 analogs of Table 4

| Compound | Structure | Supplier code |
|---|---|---|
| MAC13772 | 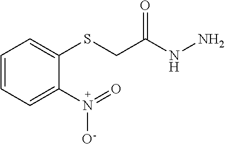 | Ryan Scientific DSHS 00862SC |
| 1a | 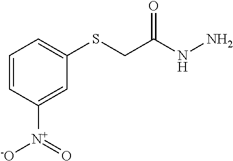 | Ryan Scientific CAS36107-14-9 |
| 1b | 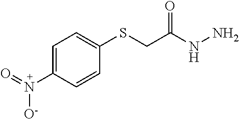 | Ambinter (France) Amb 8998045 |
| 1c | 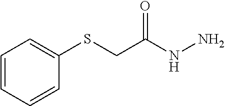 [SMILES: NNC(=O)CSc1ccccc1] | Ambinter (France) Amb 4065797 |
| 1d | 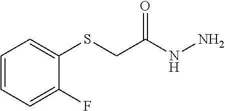 [SMILES: NNC(=O)CSc1ccccc1F] | Ambinter (France) Amb 2336529 |
| 1e | 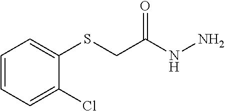 [SMILES: NNC(=O)CSc1ccccc1Cl] | Ambinter (France) Amb 8858487 |
| 1f | 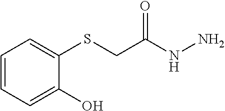 [SMILES: NNC(=O)CSc1ccccc1O] | UORSY BBV-37826735 |
| 1g | 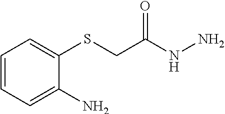 [SMILES: NNC(=O)CSc1ccccc1N] | Ambinter (France) Amb 17305609 |
| 1h | 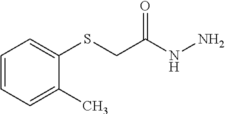 | Ambinter (France) Amb 4074830 |
| 1i | 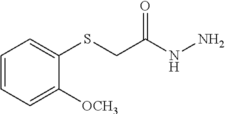 [SMILES: COc1ccccc1SCC(=O)NN] | UORSY BBV-37662617 |
| 1j | 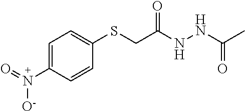 | Ambinter (France) Amb 7951732 |
| 1k | 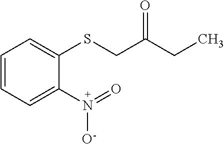 | UORSY BBV-33761526 |
| 1l | 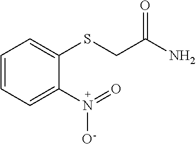 | Ambinter (France) Amb 2795194 |
| 1m | 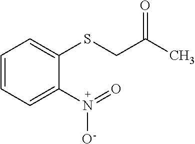 | Ambinter (France) Amb 2624964 |
| 1n | 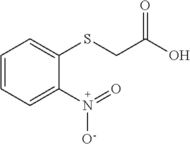 | Ambinter (France) Amb 436644 |
| 2a | 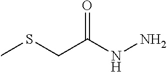 | UORSY BBV-39135451 |
| 2b | 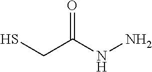 | UORSY BBV-37616329 |
| 2c | 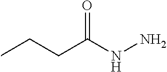 | Ambinter (France) Amb 6366352 |
| 2d | 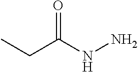 | Ambinter (France) Amb 1104708 |
| 2e | 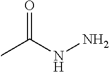 | Ambinter (France) Amb 1153969 |
| 2f | 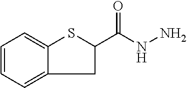 [SMILES: NNC(=O)C1Cc2ccccc2S1] | Ambinter (France) Amb 8858703 |
| 2g | 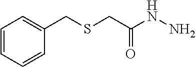 [SMILES: NNC(=O)CSCc1ccccc1] | Ambinter (France) Amb 6550464 |
| 2h | 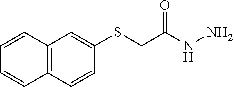 [SMILES: NNC(=O)CSc1ccc2ccccc2c1] | Ambinter (France) Amb 4074831 |
| 2i | 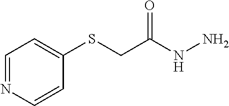 [SMILES: NNC(=O)CSc1ccncc1] | Ambinter (France) Amb 8998355 |
| 2j | 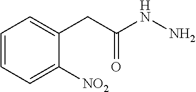 [SMILES: NNC(=O)Cc1ccccc1[N+](=O)[O-]] | Ambinter (France) Amb 6739870 |

Initially, changes of the substituents on the benzyl ring in the parent molecule were evaluated as well as their position relative to the thioacetohydrazine chain (analogs 1a through 1i). All the compounds in this category were active against BioA. However, the different modifications had a more drastic effect on their antibacterial activity against E. coli. Specifically, the position of the nitro group on the benzyl ring greatly influences biological activity with the ortho-position being highly favored (analogs 1a, 1b and 1c). Alternatively, a chloro or a methyl substitution at the ortho-position on the benzyl ring does not gravely alter antibacterial activity (analogs 1e and 1h). The requirement of the hydrazine moiety for the activity of MAC-0013772 was tested by either protecting it with an acetyl group or by modifying it (analogs 1j through 1n). Analogs lacking the hydrazine group were completely inactive in both antibacterial and biochemical assays. Given this observation, the activity of the side chain of varying lengths without the benzyl ring was tested (analogs 2a through 2e). The varying hydrazine-containing side chains only showed slight to moderate in vitro inhibition of BioA and no significant antibacterial activity. Interestingly, even in the case of the compounds 2a and 2e that had diminished antibacterial activity, growth inhibition was not suppressed in the presence of biotin.

The antibacterial activity of these 24 analogs was tested against M. smegmatis and it was found that several of the analogs had potencies similar to that of MAC-0013772, namely compounds 1b, 1c, 1d, 1e, 1h, 2g and 2i (Table 6).

TABLE 6
Activity of MAC-0013772 and analogs against Mycobacteria smegmatis

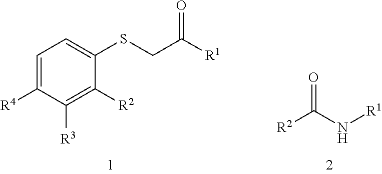

| | | | | | MIC (μg/ml)^{a} | |
|---|---|---|---|---|---|---|
| Compound | R¹ | R² | R³ | R⁴ | −bio | +bio |
| MAC13772 | NHNH₂ | NO₂ | H | H | 4 | >256 |

*Effect of Substitution on Benzene ring in Series 1

| | | | | | | |
|---|---|---|---|---|---|---|
| 1a | NHNH₂ | H | NO₂ | H | 16 | >256 |
| 1b | NHNH₂ | H | H | NO₂ | 4 | >256 |
| 1c | NHNH₂ | H | H | H | 4 | >256 |
| 1d | NHNH₂ | F | H | H | 4 | >256 |
| 1e | NHNH₂ | Cl | H | H | 4 | >256 |
| 1f | NHNH₂ | OH | H | H | 32 | 256 |
| 1g | NHNH₂ | NH₂ | H | H | 32 | 256 |
| 1h | NHNH₂ | CH₃ | H | H | 4 | >256 |
| 1i | NHNH₂ | OCH₃ | H | H | 16 | >256 |

*Effect of changing hydrazine functionality on R¹ in Series 1

| | | | | | | |
|---|---|---|---|---|---|---|
| 1j | NHNHAc | H | H | NO₂ | >256 | >256 |
| 1k | CH₂CH₃ | NO₂ | H | H | >256 | >256 |
| 1l | NH₂ | NO₂ | H | H | 128 | >256 |
| 1m | CH₃ | NO₂ | H | H | 256 | 256 |
| 1n | OH | NO₂ | H | H | >256 | >256 |

*Activity of the side chain of the parent molecule

| | | | | | | |
|---|---|---|---|---|---|---|
| 2a | NH₂ | CH₂SCH₃ | — | — | 32 | 32 |
| 2b | NH₂ | CH₂SH | — | — | 32 | 32 |
| 2c | NH₂ | CH₂CH₂CH₃ | — | — | 64 | 64 |
| 2d | NH₂ | CH₂CH₃ | — | — | 64 | 64 |
| 2e | NH₂ | CH₃ | — | — | >256 | >256 |

*Effect of different ring substituents in R² in Series 2^{b}

| | | | | | | |
|---|---|---|---|---|---|---|
| 2f | NH₂ | Benthiophene | — | — | 64 | >256 |
| 2g | NH₂ | CH₂SCH₂Ph | — | — | 8 | >256 |
| 2h | NH₂ | CH₂SNaph | — | — | 32 | >256 |
| 2i | NH₂ | CH₂SPyr | — | — | 8 | 128 |
| 2j | NH₂ | Nitrobenzyl | — | — | 256 | >256 |

^{a}MICs are determined against E. coli MG1655 in absence and presence of 2 nM of biotin
^{b}Abbreviations: Benthiophene: benzothiophene; Ph: phenyl; Naph: naphthalenyl; Pyr: pyridine

### Example 3

The activity of MAC13772 was tested against a number of organisms in addition to Escherichia coli using methods similar to those described in Example 2.

The results show that this compound is active against Salmonella typhimurium, Klebsiella pneumonia, Acinetobacter baumannii, Bacillus subtilis, Staphylococcus aureus and Mycobacterium tuberculosis.

1. A compound of formula (III) for use as an anti-bacterial agent, 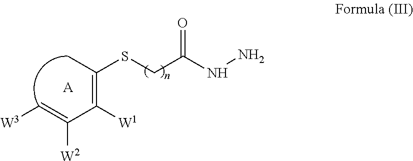
wherein
A is a ring selected from the group consisting of phenyl, pyridinyl, naphthanyl, quinoline or indole;
W¹, W² and W³ are independently selected from is H, OH, NO₂, NH₂, halogen, C₁-C₆ alkyl, C₁-C₆ alkyl halide, carboxyl, acyl halide, COW, wherein R^{a} and R^{b} are independently selected from C₁-C₆ alkyl; and
n is 0-5.

2. The compound for use as defined in claim 1, wherein the compound has the following formula: 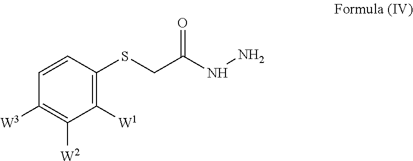
wherein W¹, W² and W³ are as defined.

3. The compound for use as defined in claim 1, selected from the group consisting of 2-(2-nitrophenylthio) acetohydrazide, 2-(3-nitrophenylthio)acetohydrazide, 2-(4-nitrophenylthio)-acetohydrazide, 2-(phenylthio)acetohydrazide, 2-(2-fluorophenylthio)acetohydrazide, 2-(2-chlorophenylthio) acetohydrazide, 2-(2-hydroxyphenylthio)acetohydrazide, 2-(2-aminophenylthio)acetohydrazide, 2-(o-tolylthio)acetohydrazide, 2-(2-methoxyphenylthio)-acetohydrazide, 2,3-dihydrobenzo[b]thiophene-2-carbohydrazide, 2-(benzylthio)acetohydrazide, 2-(pyridin-4-ylthio)acetohydrazide and 2-(naphthalen-2-ylthio)acetohydrazide.

4. The compound for use as defined in claim 2, wherein one of W¹, W² and W³ is OH, NO₂, NH₂, halogen, C₁-C₆ alkyl, C₁-C₆ alkyl halide, carboxyl, acyl halide, COR^{a}, wherein R^{a} and R^{b} are independently selected from C₁-C₆ alkyl, and the other two of W¹, W² and W³ are each H.

5. A method of inhibiting bacteria comprising exposing the bacteria to a compound as defined in claim 1

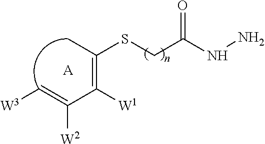

6. The method of claim 5, wherein the bacteria are selected from the group consisting of Escherichia coli, Enterococci such as ENTEROCOCCUS FAECALIS and ENTEROCOCCUS FAECIUM, Streptococcus such as S. pneumoniae, S. viridans S. pyogenes and S. pharyngitis, Staphylococcus such as S aureus, Pseudomonas such as P. aeruginosa and P. syringae, Salmonella such as S. enterica, S. typhi and S. panama, Mycobacteria such as M. tuberculosis, M. bovis, M. africanum, M. microti and M. leprae, Acinebacter such as Acinetobacter baumannii and Klebsiella such as Klebsiella pneumonia, K. granulomatis and K. planticola.

7. The method of claim 5, wherein the compound exhibits a minimal inhibitory concentration of less than 50 μg/ml.

8. The method of claim 5, wherein the compound inhibits biotin synthesis.

9. The method of claim 5, wherein the compound has the following formula: 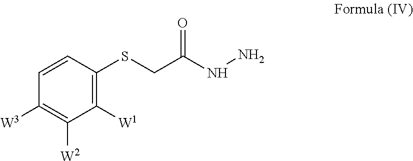
wherein W¹, W² and W³ are as defined.

10. A compound of formula (I) for use as an anti-bacterial agent, 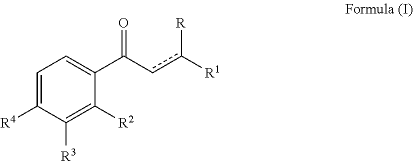
wherein
R and R¹ are independently selected from H, C₁-C₆ alkyl, C₁-C₆ alkyl halide, halogen (e.g. Br, Cl, F and I), hydroxyl, thiol, carboxyl, acyl halide (—CO-halogen), alkanoyl (—COR^{a}), —OR^{a}, —NH₂, —NO₂, —NR^{a}R^{b} or —SR^{a}, wherein R^{a} and R^{b} are independently selected from C₁-C₆ alkyl; and
R², R³ and R⁴ are independently selected from H, C₁-C₆ alkyl, C₁-C₆ alkyl halide, halogen, hydroxyl, thiol, carboxyl, acyl halide, alkanoyl (—COR^{a}), —OR^{a}, —NH₂, —NO₂, NHR^{a}, —NR^{a}R^{b} or —SR^{a}, wherein R^{a} and R^{b} are independently selected from C₁-C₆ alkyl.

11. The compound for use as defined in claim 10, wherein R and R¹ are the same substituent, and one of R², R³ and R⁴ is C₁-C₆ alkyl, C₁-C₆ alkyl halide, halogen, hydroxyl, thiol, carboxyl, acyl halide, alkanoyl (—COR^{a}), —OR^{a}, —NH₂, —NO₂, —NHR^{a}, —NR^{a}R^{b} or —SR^{a}, while the other two of R², R³ and R⁴ are each H.

12. The compound for use as defined in claim 10, selected from the group consisting of 3,3-dichloro-1-(3-nitrophenyl)prop-2-en-1-one, 3,3-dichloro-1-(3-nitrophenyl)propan-1-one, 1-(3-nitrophenyl)propan-1-one and 3-methyl-1-(3-nitrophenyl)butan-1-one.

13. A compound for use as an anti-bacterial agent having the following general formula 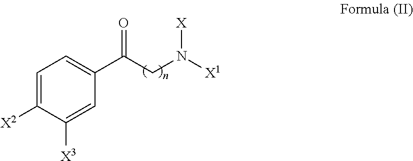
wherein
X and X¹ are independently selected from H, C₁-C₆ alkyl, C₁-C₆ alkyl halide, carboxyl, acyl halide, —COR^{a}, —COOR^{a} and C₁-C₆ alkyl-carboxyl, wherein R^{a} and R^{b} are independently selected from C₁-C₆ alkyl; or
X and X¹ together form a heterocyclic ring with N₁, wherein said ring comprises from 4-6 carbon atoms and may include a second hetero atom selected from N or S, and wherein said ring is optionally substituted with a group selected from C₁-C₆ alkyl, C₁-C₆ alkyl halide, halogen, hydroxyl, carboxyl, acyl halide, —COR^{a}, —COOR^{a} and C₁-C₆ alkyl-carboxyl, wherein R^{a} and R^{b} are independently selected from C₁-C₆ alkyl;
X² and X³ are independently selected from H, C₁-C₆ alkyl, C₁-C₆ alkyl halide, halogen, hydroxyl, thiol, carboxyl, acyl halide, —COR^{a}, —OR^{a}, —NH₂, —NO₂, —NHR^{a}, —NR^{a}R^{b} or —SR^{a}, wherein R^{a} and R^{b} are independently selected from C₁-C₆ alkyl; or
X² and X³ together form a ring, wherein said ring may be a heterocyclic ring comprising 1 or 2 hetero atoms selected from O or N, and said ring structure may be optionally substituted with a group selected from C₁-C₆ alkyl, C₁-C₆ alkyl halide, halogen, hydroxyl, carboxyl, acyl halide, —COR^{a}, —COOR^{a} and C₁-C₆ alkyl-carboxyl, wherein R^{a} and R^{b} are independently selected from C₁-C₆ alkyl; and n is 1-5.

14. The compound for use as defined in claim 13, wherein X and X¹ form a heterocyclic ring selected from the group consisting of pyrrole, pyrrolidine, pyrimidine, piperazine, piperadine, pyridine, diazine, azepane, azepine, azopane, azocane and azocine.

15. The compound for use as defined in claim 13, wherein X² and X³ form a heterocyclic ring selected from the group consisting of dioxolane, tetrahydrofuran, furan, oxane, dioxane, oxapane, oxepine, dioxapane, dioxapine, thiane, thiopyran, dithiane, dithiine, thiepane, thiolane and thiophene.

16. The compound for use as defined in claim 4, selected from the group consisting of 3-(dimethylamino)-1-(4-methoxyphenyl)propan-1-one, 1-(2,3-dihydrobenzo[b][1,4]dioxin-6-yl)-3-(dimethylamino)propan-1-one, and 1-(3,4-dihydro-2H-benzo[b][1,4]dioxepin-7-yl)-3-(piperidin-1-yl)propan-1-one).

17. A method of inhibiting bacteria comprising exposing the bacteria to a compound as defined in claim 10

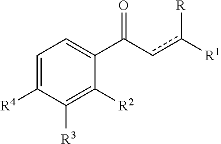

18. A method of inhibiting bacteria comprising exposing the bacteria to a compound as defined in claim 13 [chemical structure]
